(19) United States
(12) Patent Application Publication
(10) Pub. No.: US 2004/0122272 A1
(43) Pub. Date: Jun. 24, 2004

(54) Process and apparatus for removing unsaturated impurities from oxygenates to olefins streams

(76) Inventors: Cor F. Van Egmond, Pasadena, TX (US); John Richard Shutt, Tervuren (BE)

Correspondence Address: EXXONMOBIL CHEMICAL COMPANY, P O BOX 2149, BAYTOWN, TX, 77522-2149, US

(21) Appl. No.: 10/327,783

(22) Filed: Dec. 23, 2002

(51) Int. Cl. C07C 5/00

(52) U.S. Cl. 585/634

(57) ABSTRACT

Disclosed is a method and apparatus for removing highly unsaturated contaminants from an effluent stream produced by an oxygenates to olefins process. The oxygenates to olefins process produces an effluent that contains low concentrations of acetylene, methyl acetylene and propadiene. These contaminants can be removed using a “front-end” scheme, which utilizes internally generated hydrogen, to selectively hydrogenate these highly unsaturated contaminants without significant loss of olefin products.

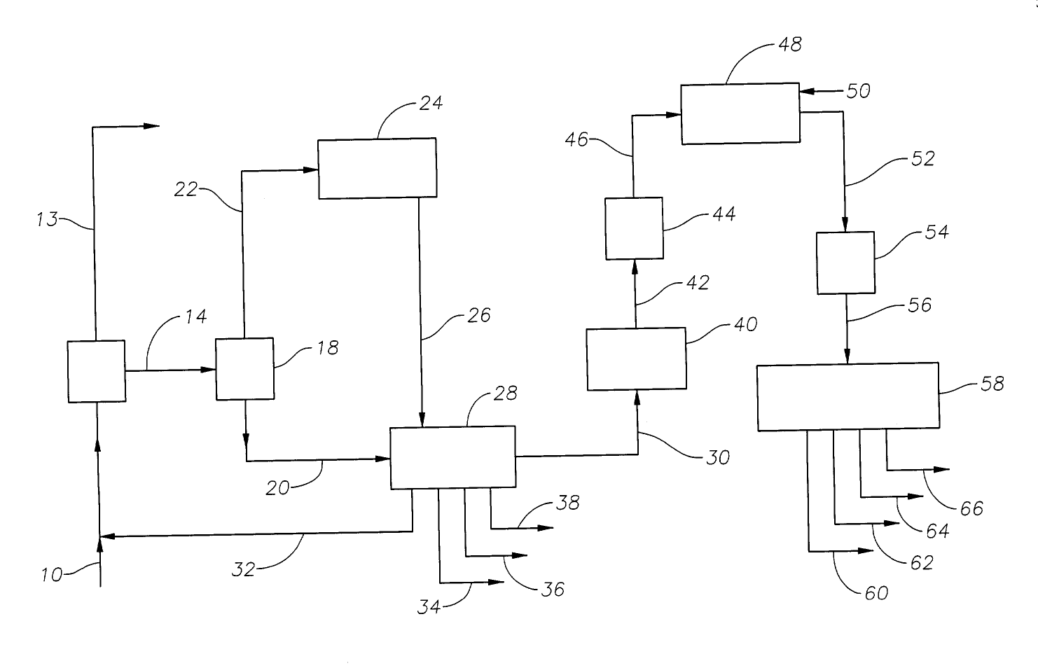

### FIELD

The present invention generally relates to a method of selectively hydrogenating highly unsaturated contaminants in an oxygenates to olefins (OTO) product stream. More particularly, this invention relates to hydrogenating acetylene, methyl acetylene, and/or propadiene in an oxygenates to olefins product stream using internally generated hydrogen.

### BACKGROUND

Making light olefins from oxygenates has become an alternative to the traditional catalytic or steam cracking processes for producing olefins. Making olefins from oxygenated feedstocks produces a unique effluent stream that must ultimately be separated and purified to produce the high purity-olefin products currently desired, e.g., mono-olefins, having a single double bond. The present invention relates to removing the highly unsaturated hydrocarbons acetylene, methyl acetylene, and/or propadiene from the effluent of an oxygenates to olefins process by selective hydrogenation. These compounds poison polyolefin catalysts, and therefore must be almost completely removed from olefin product streams. For ethylene, current manufacturing specifications call for acetylene levels to be under 0.5 mole ppm. For propylene, current manufacturing specifications call for methyl acetylene and propadiene levels to be under 2.9 mole ppm.

Catalysts for selectively hydrogenating highly unsaturated compounds are known in the art. For example, U.S. Pat. No. 6,084,140 to Kitamura et al. discloses a palladium and alumina catalyst for hydrogenating highly unsaturated hydrocarbons in olefin streams from steam cracking processes. The catalyst can hydrogenate acetylene, methyl acetylene, and propadiene, with only limited hydrogenation of the olefin products.

In steam cracking, there are two general types of selective hydrogenation processes that are used for removing highly unsaturated contaminants from hydrocarbon streams. The first type is known as “front-end” hydrogenation, which involves passing a hydrogen-containing hydrocarbon process stream over a hydrogenation catalyst, with no externally added hydrogen required for the hydrogenation process. The amount of hydrogen in the hydrocarbon process stream must be sufficient to hydrogenate the unsaturated contaminants, but should not be so great that excessive hydrogenation of olefin products occurs. Front end converters are thus hydrogenation reactors that use the hydrogen inherently present in a feed stream, i.e., the hydrogen by-product from a main conversion reactor, e.g., a cracking furnace or OTO fluid bed reactor. Front-end hydrogenation thus typically occurs in a hydrogenation reactor or converter located at the front-end of the olefins plant, somewhere between compression and cold-fractionation treatment.

The second type of selective hydrogenation process is known as “tail-end” or “back-end” hydrogenation. U.S. Pat. No. 4,367,353 to Inglis discusses a tail-end hydrogenation process using a supported palladium catalyst. Tail end hydrogenation involves fractionating the hydrocarbon streams away from the acetylene, propadiene or methyl acetylene before hydrogenating. Hydrogen is removed during the fractionating process and therefore, hydrogen must be re-added during the hydrogenation step. The tail end process allows for greater control of the hydrogenation process, but requires the addition of hydrogen to the process. Further, catalyst deactivation from the formation of polymers on the catalyst is of greater concern in the tail-end configuration than in the front-end configuration. Despite its added complexity, tail-end hydrogenation is currently favored in stream cracking processes because of the extremely low allowable levels of acetylene, methyl acetylene, and propadiene in the industry. For the purposes of the present invention, a front-end converter relates to a hydrogenation reactor utilizing internal by-product hydrogen and no externally supplied hydrogen, while a tail-end converter relates to a hydrogenation reactor which utilizes an external source of hydrogen as its primary hydrogen source.

The concentrations of acetylene, methyl acetylene, and propadiene increase to about three times their initial amounts during the purification of the hydrocarbons by fractionation. This means that the concentrations of acetylene, methyl acetylene and propadiene must be about three times lower following front-end hydrogenation than in tail-end hydrogenation. However, achieving this greater purity will result in greater loss of olefin products by their saturation to alkanes during the hydrogenation process.

Accordingly, it would be desirable to provide a method to remove the small concentrations of acetylene, methyl acetylene, and propadiene from the effluent of an oxygenates to olefins reactor. The small concentration of these highly hydrogenated compounds allows for less severe hydrogenation conditions, which minimizes the loss of olefin products, while still obtaining a high purity olefin product. Thus an opportunity exists to treat oxygenates to olefins reactor effluents to provide effective hydrogenation of the lower amounts of alkynes produced while minimizing the need for externally supplied hydrogen, and avoiding over-hydrogenating the alkynes to undesired alkanes.

U.S. Pat. No. 6,049,017 to Vora et al. discloses a method for enhanced production of light olefins wherein undesired diolefins such as butadiene are removed by selective hydrogenation over a catalyst containing nickel and noble metal. Vora et al. utilize a separate hydrogen feed to achieve butadiene removal.

U.S. Pat. No. 5,877,363 to Gildert et al. discloses a process for removing alkynes (vinylacetylene, ethylacetylene) and 1,2-butadiene from a stream containing C₄ aliphatic hydrocarbons, by feeding the stream to a distillation column reactor containing a bed of hydrogenation catalyst (Pt, Pd, Rh and mixtures thereof, e.g., 0.5 wt % Pd on alumina) in the presence of hydrogen provided as necessary, and removing a C₄ stream as overhead which has reduced acetylenes and 1,2-butadiene content.

U.S. Pat. No. 4,409,410 to Cosyns et al. discloses a process for selectively hydrogenating a diolefin in a mixture of C₄₊ hydrocarbons comprising 1-olefin, by reacting the mixture with hydrogen in the presence of a catalyst comprising palladium and alumina.

U.S. Pat. No. 6,388,150 to Overbeek et al. discloses a selective hydrogenation process for mono-olefinic feeds such as those obtained by pyrolysis. The feeds contain acetylene compounds and/or dienes and are selectively hydrogenated by contacting with a selective hydrogenation catalyst on a particulate support, e.g., palladium-silver catalyst on alumina, which itself is supported on a mesh-like structure.

U.S. Pat. No. 6,303,841 to Senetar et al. teaches a process for producing ethylene wherein an oxygenate conversion effluent, treated to remove oxygenate, carbon dioxide and water, is further treated to remove hydrogen, carbon monoxide, methane, acetylene, ethylene and ethane as an overhead. The overhead is passed to a compression and selective hydrogenation zone to saturate acetylene, thereby providing a stream containing less than 1 wppm acetylene which is passed to a column operating at a temperature above −45° C. to provide a C₂ stream and an overhead comprising hydrogen and methane which streams are subsequently treated.

U.S. Pat. No. 6,486,369 to Voight et al. discloses a process for selectively hydrogenating a C₂ and C₃ olefinic feed stream containing acetylenic and diolefinic impurities whereby the acetylenes and diolefins impurities are selectively hydrogenated concurrently in a vapor phase process without first separating the C₂ and C₃ olefinic gases in separate streams. The process separates light-end gases such as hydrogen, CO and methane from the C₂ and C₃ olefinic feed stream prior to hydrogenating with externally added hydrogen.

Given the economic advantages derived from producing ethylene and propylene from oxygenates, it would be especially desirable to provide olefins pure enough to use as polymerization feedstock, while minimizing the process steps required for treating OTO effluents. It would be particularly desirable to provide a process which uses internally generated hydrogen to effect hydrogenation of highly unsaturated impurities found in OTO effluent streams.

### SUMMARY

In one aspect, the present invention relates to a method for removing acetylene from an olefinic stream, comprising: fractionating the olefinic stream comprising C₂ to C₄ olefin, hydrogen and acetylene, in a fractionator to provide a C₃ overhead stream comprising ethylene, propylene, hydrogen, CO and acetylene; directing the C₃ overhead stream to an inlet of a hydrogenation reactor and contacting the C₃ overhead stream with a hydrogenation catalyst under conditions sufficient to hydrogenate substantially all of the acetylene to olefin without substantially converting the ethylene and/or the propylene; and removing a purified olefin stream from the hydrogenation reactor. By “substantially all” is meant that at least about 90%, typically at least about 95%, e.g., at least about 99%, or even at least about 99.9% of the acetylene is hydrogenated to olefin.

In one embodiment of this aspect of the invention, the C₃ overhead stream directed to the hydrogenation reactor inlet has a temperature ranging from about 110° to about 250° F. Typically, the C₃ overhead stream directed to the inlet has a temperature ranging from about 160° to about 210° F.

In another embodiment, the hydrogenation reactor is operated at conditions comprising from about 9000 to about 25000 weight hourly space velocity and from about 150 to about 500 psig.

In still another embodiment, the C₃ overhead stream directed to the inlet comprises from about 100 ppm to about 2000 ppm CO, from about 0.1 ppm to about 40 ppm acetylene, from about 0 ppm to about 80 ppm propadiene, and from about 0 ppm to about 80 ppm methyl acetylene.

In yet another embodiment, the hydrogenation reactor is operated at conditions comprising from about 10000 to about 18000 weight hourly space velocity and from about 250 to about 450 psig.

In still yet another embodiment, the C₃ overhead stream directed to the inlet comprises from about 200 ppm to about 400 ppm CO, from about 0.1 ppm to about 10 ppm acetylene, from about 0 ppm to about 40 ppm propadiene, and from about 0 to about 40 ppm methyl acetylene.

In yet still another embodiment, the C₃ overhead stream has a molar ratio of carbon monoxide/acetylene ranging from about 100 to about 20, e.g., ranging from about 80 to about 40.

In another embodiment, the fractionating takes place in a fractionating tower which separates C₃ hydrocarbons from dimethyl ether and heavier boiling materials. Typically, such fractionating takes place in a deetherizer, a depropanizer, a depropylenizer, and/or a C₃ splitter.

In yet another embodiment, at least about 95% of the acetylene is converted in the hydrogenation reactor, typically at least about 99% of the acetylene being so converted.

In still another embodiment, the C₃₋ overhead stream directed to said inlet comprises acetylene, methyl acetylene and propadiene. Typically, at least about 95% of the acetylene, at least about 60% of the methyl acetylene and at least about 20% of the propadiene are converted in the hydrogenation reactor, say, at least about 99% of the acetylene, at least about 80% of the methyl acetylene and at least about 25% of the propadiene are converted.

In yet still another embodiment, an effluent from the hydrogenation reactor is directed to a demethanizer which removes hydrogen, carbon monoxide and methane from the effluent to provide a demethanizer product effluent.

In still yet another embodiment, the demethanizer product effluent is directed to a C₂ splitter to provide an ethylene product stream comprising less than about 0.3 vppm (parts per million by volume) acetylene.

In another embodiment of this aspect of the invention, the demethanizer product effluent is directed to a C₃ splitter to provide a propylene product stream comprising less than about 2.0 vppm acetylene, less than about 3.0 vppm methyl acetylene and less than about 3.0 vppm propadiene.

In still another embodiment, the olefinic stream contains an ether impurity, e.g., dimethyl ether, and is treated with a deetherizer to at least partially remove the ether impurity prior to the fractionating.

In yet another embodiment, the olefin stream from the hydrogenation reactor contains water and is directed to a molecular sieve dryer which provides a dried olefin stream from which water is at least partially removed. Such a dryer utilizes a molecular sieve having a pore size of suitable for removal of water and methanol has a pore size of at least about 3.0 angstroms in diameter and is well known to those of skill in the art.

In another embodiment, the olefin stream from the hydrogenation reactor contains water and methanol and is directed to a molecular sieve dryer which provides a dried olefin stream from which water and methanol are at least partially removed. Such a dryer utilizes a molecular sieve having a pore size of suitable for removal of water and methanol has a pore size of at least about 3.6 angstroms in diameter and is well known to those of skill in the art.

In yet still another embodiment, the hydrogenation catalyst comprises a metal selected from the group consisting of Ni, Pd and Pt, typically Pd. The hydrogenation catalyst can further comprise a metal selected from the group consisting of Cu, Ag and Au.

In yet another embodiment, the hydrogenation catalyst comprises an inorganic oxide support, e.g., alumina.

In still another embodiment, the hydrogenation catalyst comprises palladium and silver, supported on calcium carbonate.

In still yet another embodiment, the hydrogenation catalyst comprises palladium supported on alumina.

In another embodiment, the hydrogenation catalyst comprises from about 0.001 to about 2 wt % of the hydrogenation metal, say, from about 0.01 to about 1 wt % palladium.

In still another embodiment, external hydrogen is added to the hydrogenation reactor.

In yet another embodiment, no external hydrogen is added to the hydrogenation reactor.

In another aspect, the present invention relates to a method for converting oxygenates to olefins which comprises: a) contacting an oxygenates feed in an oxygenates to olefins reactor with an oxygenates to olefins catalyst under conditions sufficient to provide an oxygenates to olefins product stream comprising ethylene, propylene, C₄ olefin, hydrogen, carbon monoxide, and acetylene; b) fractionating the oxygenates to olefins product stream to provide a fractionated overhead stream comprising ethylene, propylene, hydrogen, from about 500 ppm to about 1200 ppm CO, from about 0.2 ppm to about 15 ppm acetylene, from about 0 ppm to about 40 ppm propadiene, and from about 0 to about 40 ppm methyl acetylene; c) hydrogenating the fractionated overhead stream by contacting with a hydrogenation catalyst in a hydrogenation reactor under conditions sufficient to partially hydrogenate the acetylene, without substantially hydrogenating the ethylene and the propylene; and d) removing a purified olefin stream from the hydrogenation reactor.

In one embodiment of this aspect of the present invention, the fractionated overhead stream comprises from about 100 ppm to about 400 ppm CO, from about 0.1 ppm to about 10 ppm acetylene, from about 0 ppm to about 40 ppm propadiene, and from about 0 to about 40 ppm methyl acetylene.

In another embodiment, the fractionated overhead stream has a molar ratio of carbon monoxide/acetylene ranging from about 100 to about 20, say, ranging from about 80 to about 40.

In yet another embodiment, the fractionated overhead stream comprises propane.

In still another embodiment, the fractionated overhead stream hydrogenated by the hydrogenation reactor has a temperature ranging from about 110° to about 250° F., say, from about 160° to about 210° F.

In still yet another embodiment, the hydrogenation reactor is operated at conditions comprising from about 9000 to about 25000 weight hourly space velocity and from about 150 to about 500 psig, say, from about 10000 to about 18000 weight hourly space velocity and from about 250 to about 450 psig.

In yet still another embodiment, the fractionating takes place in a fractionating tower which separates C₃ hydrocarbons from dimethyl ether and heavier boiling materials.

In another embodiment, the fractionating takes place in a deetherizer, depropanizer, or depropylenizer.

In still another embodiment, the purified olefin stream from the hydrogenation reactor is directed to a molecular sieve dryer which provides a dried olefin stream from which water is at least partially removed.

In still another embodiment, the purified olefin stream from the hydrogenation reactor contains water and methanol and is directed to a molecular sieve dryer which provides a dried olefin stream from which water and methanol are at least partially removed.

In yet another embodiment, the dried olefin stream is cryogenically processed to provide a C₂ and C₃ fuel stream, a C₁ and hydrogen tail gas stream, an ethylene product stream and a propylene product stream.

In still another embodiment, the ethylene product stream comprises less than about 0.3 vppm acetylene.

In yet still another embodiment, the propylene product stream comprises less than about 2.0 vppm acetylene, less than about 3.0 vppm methyl acetylene and less than about 3.0 vppm propadiene.

In another embodiment, the hydrogenation catalyst comprises a metal selected from the group consisting of Ni, Pd and Pt, typically palladium. The hydrogenation catalyst can further comprise a metal selected from the group consisting of Cu, Ag and Au.

In still another embodiment, the hydrogenation catalyst comprises an inorganic oxide support, e.g., alumina.

In yet another embodiment, the hydrogenation catalyst comprises palladium and silver, supported on calcium carbonate.

In yet still another embodiment, the hydrogenation catalyst comprises palladium supported on alumina.

In still yet another embodiment, the hydrogenation catalyst comprises from about 0.001 to about 2 wt % of the hydrogenation metal, e.g., from about 0.01 to about 1 wt % palladium.

In another embodiment, external hydrogen is added to the hydrogenation reactor.

In yet another embodiment, no external hydrogen is added to the hydrogenation reactor.

In still another aspect of the invention, the oxygenates to olefins catalyst comprises a molecular sieve.

In yet still another aspect, the molecular sieve has a pore diameter of less than 5.0 Angstroms. Typically, the molecular sieve is selected from the group consisting of AEI, AFT, APC, ATN, ATT, ATV, AWW, BIK, CAS, CHA, CHI, DAC, DDR, EDI, ERI, GOO, KFI, LEV, LOV, LTA, MON, PAU, PHI, RHO, ROG, THO, ALPO-18, ALPO-34, SAPO-17, SAPO-18, SAPO-34, and substituted groups thereof, e.g., the molecular sieve is at least one selected from the group consisting of ALPO-18, ALPO-34, SAPO-17, SAPO-18, and SAPO-34.

In another embodiment, the molecular sieve has a pore diameter of 5-10 Angstroms. Typically, the molecular sieve is selected from the group consisting of MFI, MEL, MTW, EUO, MTT, HEU, FER, AFO, AEL, TON, and substituted groups thereof.

In yet another aspect, the present invention relates to an apparatus for converting oxygenates to an olefins stream containing C₂ to C₄ olefins and acetylene as an impurity, and providing a purified ethylene and/or propylene stream proportionally reduced in the impurity content, the apparatus comprising: i) an oxygenates to olefins reactor comprising a fluidized bed which comprises an oxygenates to olefins catalyst, the reactor further comprising an inlet for oxygenate feed and an outlet for the olefins stream; ii) a fractionator for separating from the olefins stream a bottoms stream containing unreacted oxygenate, C₄₊ hydrocarbons and waste water, and an overheads stream comprising ethylene, propylene, hydrogen, acetylene and CO; iii) a hydrogenation reactor for hydrogenating the overheads stream by contacting with a hydrogenation catalyst under conditions sufficient to partially hydrogenate the acetylene, without substantially hydrogenating the ethylene and the propylene, to provide a purified stream of reduced acetylene content; and iv) a means for cryogenically fractionating the purified stream to provide a purified ethylene product and a purified propylene product. In one embodiment of this aspect of the invention, the fractionator is a fractionating tower which separates C₃ hydrocarbons from dimethyl ether and heavier boiling materials. Typically, such a fractionator is selected from the group consisting of deetherizer, depropanizer, depropylenizer, and C₃ splitter.

In still another embodiment, the fractionating takes place in a deetherizer fractionating tower which separates C₃ hydrocarbons from dimethyl ether and heavier boiling materials.

In another embodiment, the fractionating takes place in a depropanizer fractionating tower, which separates C₃ hydrocarbons and dimethyl ether from C₄ and heavier boiling materials.

In yet another embodiment, the fractionating takes place in a depropylenizer fractionating tower, which separates C₃⁼ and lighter boiling materials from propane and heavier boiling materials.

In another embodiment, the apparatus of the invention further comprises a means for quenching the olefins stream to provide a quenched olefins stream.

In yet another embodiment, the apparatus of the invention further comprises a means for compressing the quenched olefins stream to provide a compressed, quenched olefins stream.

In still another embodiment, the apparatus of the invention further comprises a caustic treater for treating the overheads stream to remove carbon dioxide from the overheads stream to provide a caustic-treated stream.

In still yet another embodiment, the apparatus of the invention further comprises a molecular sieve dryer upstream from the hydrogenation reactor, to remove water from the caustic-treated stream.

In another embodiment, the apparatus of the invention further comprises a molecular sieve dryer downstream from the hydrogenation reactor, to remove water from the purified stream of reduced acetylene content.

In another embodiment, the apparatus of the invention further comprises a molecular sieve dryer downstream from the hydrogenation reactor, to remove water and methanol from the purified stream of reduced acetylene content.

### BRIEF DESCRIPTION OF THE DRAWINGS

The invention will be better understood by reference to the Detailed Description when taken together with the attached drawing, wherein:

The FIGURE is a flow diagram of an embodiment of the invention providing a hydrogenation reactor for treating overhead of a fractionating tower which separates C₃ and lower boiling hydrocarbons from dimethyl ether and heavier boiling materials.

### DETAILED DESCRIPTION

Molecular Sieves and Catalysts Thereof for Use in OTO Conversion

Molecular sieves suited to use in the present invention for converting oxygenates to olefins have various chemical and physical, framework, characteristics. Molecular sieves have been well classified by the Structure Commission of the International Zeolite Association according to the rules of the IUPAC Commission on Zeolite Nomenclature. A framework-type describes the connectivity, topology, of the tetrahedrally coordinated atoms constituting the framework, and making an abstraction of the specific properties for those materials. Framework-type zeolite and zeolite-type molecular sieves for which a structure has been established, are assigned a three letter code and are described in the Atlas of Zeolite Framework Types, 5th edition, Elsevier, London, England (2001), which is herein fully incorporated by reference.

Non-limiting examples of these molecular sieves are the small pore molecular sieves of a framework-type selected from the group consisting of AEI, AFT, APC, ATN, ATT, ATV, AWW, BIK, CAS, CHA, CHI, DAC, DDR, EDI, ERI, GOO, KFI, LEV, LOV, LTA, MON, PAU, PHI, RHO, ROG, THO, and substituted forms thereof, the medium pore molecular sieves of a framework-type selected from the group consisting of AFO, AEL, EUO, HEU, FER, MEL, MFI, MTW, MTT, TON, and substituted forms thereof; and the large pore molecular sieves of a framework-type selected from the group consisting of EMT, FAU, and substituted forms thereof. Other molecular sieves have a framework-type selected from the group consisting of ANA, BEA, CFI, CLO, DON, GIS, LTL, MER, MOR, MWW and SOD. Non-limiting examples of the preferred molecular sieves, particularly for converting an oxygenate containing feedstock into olefin(s), include those having a framework-type selected from the group consisting of AEL, AFY, BEA, CHA, EDI, FAU, FER, GIS, LTA, LTL, MER, MFI, MOR, MTT, MWW, TAM and TON. In one preferred embodiment, the molecular sieve of the invention has an AEI topology or a CHA topology, or a combination thereof, most preferably a CHA topology.

Molecular sieve materials all have 3-dimensional, four-connected framework structure of corner-sharing TO₄ tetrahedra, where T is any tetrahedrally coordinated cation. These molecular sieves are typically described in terms of the size of the ring that defines a pore, where the size is based on the number of T atoms in the ring. Other framework-type characteristics include the arrangement of rings that form a cage, and when present, the dimension of channels, and the spaces between the cages. See van Bekkum, et al., Introduction to Zeolite Science and Practice, Second Completely Revised and Expanded Edition, Volume 137, pages 1-67, Elsevier Science, B. V., Amsterdam, Netherlands (2001).

The small, medium and large pore molecular sieves have from a 4-ring to a 12-ring or greater framework-type. In a preferred embodiment, the zeolitic molecular sieves have 8-, 10- or 12-ring structures or larger and an average pore size in the range of from about 3 Å to 15 Å. In the most preferred embodiment, the molecular sieves of the invention, preferably silicoaluminophosphate molecular sieves have 8-rings and an average pore size less than about 5 Å, preferably in the range of from 3 Å to about 5 Å, more preferably from 3 Å to about 4.5 Å, and most preferably from 3.5 Å to about 4.2 Å.

Molecular sieves, particularly zeolitic and zeolitic-type molecular sieves, preferably have a molecular framework of one, preferably two or more corner-sharing [TO₄] tetrahedral units, more preferably, two or more [SiO₄], [AlO₄] and/or [PO₄] tetrahedral units, and most preferably [SiO₄], [AlO₄] and [PO₄] tetrahedral units. These silicon, aluminum, and phosphorous based molecular sieves and metal containing silicon, aluminum and phosphorous based molecular sieves have been described in detail in numerous publications including for example, U.S. Pat. No. 4,567,029 (MeAPO where Me is Mg, Mn, Zn, or Co), U.S. Pat. No. 4,440,871 (SAPO), European Patent Application EP-A-0 159 624 (ELAPSO where E1 is As, Be, B, Cr, Co, Ga, Ge, Fe, Li, Mg, Mn, Ti or Zn), U.S. Pat. No. 4,554,143 (FeAPO), U.S. Pat. Nos. 4,822,478, 4,683,217, 4,744,885 (FeAPSO), EP-A-0 158 975 and U.S. Pat. No. 4,935,216 (ZnAPSO, EP-A-0 161 489 (CoAPSO), EP-A-0 158 976 (ELAPO, where EL is Co, Fe, Mg, Mn, Ti or Zn), U.S. Pat. No. 4,310,440 (AlPO₄), EP-A-0 158 350 (SENAPSO), U.S. Pat. No. 4,973,460 (LiAPSO), U.S. Pat. No. 4,789,535 (LiAPO), U.S. Pat. No. 4,992,250 (GeAPSO), U.S. Pat. No. 4,888,167 (GeAPO), U.S. Pat. No. 5,057,295 (BAPSO), U.S. Pat. No. 4,738,837 (CrAPSO), U.S. Pat. Nos. 4,759,919, and 4,851,106 (CrAPO), U.S. Pat. Nos. 4,758,419, 4,882,038, 5,434,326 and 5,478,787 (MgAPSO), U.S. Pat. No. 4,554,143 (FeAPO), U.S. Pat. No. 4,894,213 (AsAPSO), U.S. Pat. No. 4,913,888 (AsAPO), U.S. Pat. Nos. 4,686,092, 4,846,956 and 4,793,833 (MnAPSO), U.S. Pat. Nos. 5,345,011 and 6,156,931 (MnAPO), U.S. Pat. No. 4,737,353 (BeAPSO), U.S. Pat. No. 4,940,570 (BeAPO), U.S. Pat. Nos. 4,801,309, 4,684,617 and 4,880,520 (TiAPSO), U.S. Pat. Nos. 4,500,651, 4,551,236 and 4,605,492 (TiAPO), U.S. Pat. No. 4,824,554, 4,744,970 (CoAPSO), U.S. Pat. No. 4,735,806 (GaAPSO) EP-A-0 293 937 (QAPSO, where Q is framework oxide unit [QO₂]), as well as U.S. Pat. Nos. 4,567,029, 4,686,093, 4,781,814, 4,793,984, 4,801,364, 4,853,197, 4,917,876, 4,952,384, 4,956,164, 4,956,165, 4,973,785, 5,241,093, 5,493,066 and 5,675,050, all of which are herein fully incorporated by reference.

Other molecular sieves include those described in EP-0 888 187 B1 (microporous crystalline metallophosphates, SAPO₄ (UIO-6)), U.S. Pat. No. 6,004,898 (molecular sieve and an alkaline earth metal), U.S. patent application Ser. No. 09/511,943 filed Feb. 24, 2000 (integrated hydrocarbon co-catalyst), PCT WO 01/64340 published Sep. 7, 2001 (thorium containing molecular sieve), and R. Szostak, Handbook of Molecular Sieves, Van Nostrand Reinhold, New York, New York (1992), which are all herein fully incorporated by reference.

The more preferred silicon, aluminum and/or phosphorous containing molecular sieves, and aluminum, phosphorous, and optionally silicon, containing molecular sieves include aluminophosphate (ALPO) molecular sieves and silicoaluminophosphate (SAPO) molecular sieves and substituted, preferably metal substituted, ALPO and SAPO molecular sieves. The most preferred molecular sieves are SAPO molecular sieves, and metal substituted SAPO molecular sieves. In an embodiment, the metal is an alkali metal of Group IA of the Periodic Table of Elements, an alkaline earth metal of Group IIA of the Periodic Table of Elements, a rare earth metal of Group IIIB, including the Lanthanides: lanthanum, cerium, praseodymium, neodymium, samarium, europium, gadolinium, terbium, dysprosium, holmium, erbium, thulium, ytterbium and lutetium; and scandium or yttrium of the Periodic Table of Elements, a transition metal of Groups IVB, VB, VIB, VIIB, VIIIB, and IB of the Periodic Table of Elements, or mixtures of any of these metal species. In one preferred embodiment, the metal is selected from the group consisting of Co, Cr, Cu, Fe, Ga, Ge, Mg, Mn, Ni, Sn, Ti, Zn and Zr, and mixtures thereof. In another preferred embodiment, these metal atoms discussed above are inserted into the framework of a molecular sieve through a tetrahedral unit, such as [MeO₂], and carry a net charge depending on the valence state of the metal substituent. For example, in one embodiment, when the metal substituent has a valence state of +2, +3, +4, +5, or +6, the net charge of the tetrahedral unit is between −2 and +2.

In one embodiment, the molecular sieve, as described in many of the U.S. Patents mentioned above, is represented by the empirical formula, on an anhydrous basis:

mR:(MₓAl_{y}P_{z})O₂

wherein R represents at least one templating agent, preferably an organic templating agent; m is the number of moles of R per mole of (MₓAl_{y}P_{z})O₂ and m has a value from 0 to 1, preferably 0 to 0.5, and most preferably from 0 to 0.3; x, y, and z represent the mole fraction of Al, P and M as tetrahedral oxides, where M is a metal selected from one of Group IA, IIA, IB, IIIB, IVB, VB, VIB, VIIB, VIIIB and lanthanides of the Periodic Table of Elements, preferably M is selected from one of the group consisting of Co, Cr, Cu, Fe, Ga, Ge, Mg, Mn, Ni, Sn, Ti, Zn and Zr. In an embodiment, m is greater than or equal to 0.2, and x, y and z are greater than or equal to 0.01. In another embodiment, m is greater than 0.1 to about 1, x is greater than 0 to about 0.25, y is in the range of from 0.4 to 0.5, and z is in the range of from 0.25 to 0.5, more preferably m is from 0.15 to 0.7, x is from 0.01 to 0.2, y is from 0.4 to 0.5, and z is from 0.3 to 0.5.

Non-limiting examples of SAPO and ALPO molecular sieves of the invention include one or a combination of SAPO-5, SAPO-8, SAPO-11, SAPO-16, SAPO-17, SAPO-18, SAPO-20, SAPO-31, SAPO-34, SAPO-35, SAPO-36, SAPO-37, SAPO-40, SAPO-41, SAPO-42, SAPO-44 (U.S. Pat. No. 6,162,415), SAPO-47, SAPO-56, ALPO-5, ALPO-11, ALPO-18, ALPO-31, ALPO-34, ALPO-36, ALPO-37, ALPO-46, and metal containing molecular sieves thereof. The more preferred zeolite-type molecular sieves include one or a combination of SAPO-18, SAPO-34, SAPO-35, SAPO-44, SAPO-56, ALPO-18 and ALPO-34, even more preferably one or a combination of SAPO-18, SAPO-34, ALPO-34 and ALPO-18, and metal containing molecular sieves thereof, and most preferably one or a combination of SAPO-34 and ALPO-18, and metal containing molecular sieves thereof.

In an embodiment, the molecular sieve is an intergrowth material having two or more distinct phases of crystalline structures within one molecular sieve composition. In particular, intergrowth molecular sieves are described in the U.S. patent application Ser. No. 09/924,016 filed Aug. 7, 2001 and PCT WO 98/15496 published Apr. 16, 1998, both of which are herein fully incorporated by reference. In another embodiment, the molecular sieve comprises at least one intergrown phase of AEI and CHA framework-types. For example, SAPO-18, ALPO-18 and RUW-18 have an AEI framework-type, and SAPO-34 has a CHA framework-type.

Molecular Sieve Synthesis

The synthesis of molecular sieves is described in many of the references discussed above. Generally, molecular sieves are synthesized by the hydrothermal crystallization of one or more of a source of aluminum, a source of phosphorous, a source of silicon, a templating agent, and a metal containing compound. Typically, a combination of sources of silicon, aluminum and phosphorous, optionally with one or more templating agents and/or one or more metal containing compounds are placed in a sealed pressure vessel, optionally lined with an inert plastic such as polytetrafluoroethylene, and heated, under a crystallization pressure and temperature, until a crystalline material is formed, and then recovered by filtration, centrifugation and/or decanting.

In a preferred embodiment the molecular sieves are synthesized by forming a reaction product of a source of silicon, a source of aluminum, a source of phosphorous, an organic templating agent, preferably a nitrogen containing organic templating agent, and one or more polymeric bases. This particularly preferred embodiment results in the synthesis of a silicoaluminophosphate crystalline material that is then isolated by filtration, centrifugation and/or decanting.

Non-limiting examples of silicon sources include a silicates, fumed silica, for example, Aerosil-200 available from Degussa Inc., New York, N.Y., and CAB-O-SIL M-5, silicon compounds such as tetraalkyl orthosilicates, for example, tetramethyl orthosilicate (TMOS) and tetraethylorthosilicate (TEOS), colloidal silicas or aqueous suspensions thereof, for example Ludox-HS-40 sol available from E. I. du Pont de Nemours, Wilmington, Del., silicic acid, alkali-metal silicate, or any combination thereof. The preferred source of silicon is a silica sol.

Non-limiting examples of aluminum sources include aluminum-containing compositions such as aluminum alkoxides, for example aluminum isopropoxide, aluminum phosphate, aluminum hydroxide, sodium aluminate, pseudo-boehmite, gibbsite and aluminum trichloride, or any combinations thereof. A preferred source of aluminum is pseudo-boehmite, particularly when producing a silicoaluminophosphate molecular sieve.

Non-limiting examples of phosphorous sources, which may also include aluminum-containing phosphorous compositions, include phosphorous-containing, inorganic or organic, compositions such as phosphoric acid, organic phosphates such as triethyl phosphate, and crystalline or amorphous aluminophosphates such as AlPO₄, phosphorous salts, or combinations thereof. The preferred source of phosphorous is phosphoric acid, particularly when producing a silicoaluminophosphate.

Templating agents are generally compounds that contain elements of Group VA of the Periodic Table of Elements, particularly nitrogen, phosphorus, arsenic and antimony, more preferably nitrogen or phosphorous, and most preferably nitrogen. Typical templating agents of Group VA of the Periodic Table of elements also contain at least one alkyl or aryl group, preferably an alkyl or aryl group having from 1 to 10 carbon atoms, and more preferably from 1 to 8 carbon atoms. The preferred templating agents are nitrogen-containing compounds such as amines and quaternary ammonium compounds.

The quaternary ammonium compounds, in one embodiment, are represented by the general formula R₄N⁺, where each R is hydrogen or a hydrocarbyl or substituted hydrocarbyl group, preferably an alkyl group or an aryl group having from 1 to 10 carbon atoms. In one embodiment, the templating agents include a combination of one or more quaternary ammonium compound(s) and one or more of a mono-, di- or tri-amine.

Non-limiting examples of templating agents include tetraalkyl ammonium compounds including salts thereof such as tetramethyl ammonium compounds including salts thereof, tetraethyl ammonium compounds including salts thereof, tetrapropyl ammonium including salts thereof, and tetrabutylammonium including salts thereof, cyclohexylamine, morpholine, di-n-propylamine (DPA), tripropylamine, triethylamine (TEA), triethanolamine, piperidine, cyclohexylamine, 2-methylpyridine, N,N-dimethylbenzylamine, N,N-diethylethanolamine, dicyclohexylamine, N,N-dimethylethanolamine, choline, N,N′-dimethylpiperazine, 1,4-diazabicyclo(2,2,2)octane, N′,N′,N,N-tetramethyl-(1,6)hexanediamine, N-methyldiethanolamine, N-methyl-ethanolamine, N-methyl piperidine, 3-methyl-piperidine, N-methylcyclohexylamine, 3-methylpyridine, 4-methyl-pyridine, quinuclidine, N,N′-dimethyl-1,4-diazabicyclo(2,2,2) octane ion; di-n-butylamine, neopentylamine, di-n-pentylamine, isopropylamine, t-butyl-amine, ethylenediamine, pyrrolidine, and 2-imidazolidone.

The preferred templating agent or template is a tetraethylammonium compound, such as tetraethyl ammonium hydroxide (TEAOH), tetraethyl ammonium phosphate, tetraethyl ammonium fluoride, tetraethyl ammonium bromide, tetraethyl ammonium chloride and tetraethyl ammonium acetate. The most preferred templating agent is tetraethyl ammonium hydroxide and salts thereof, particularly when producing a silicoaluminophosphate molecular sieve. In one embodiment, a combination of two or more of any of the above templating agents is used in combination with one or more of a silicon-, aluminum-, and phosphorous-source, and a polymeric base.

Polymeric bases, especially polymeric bases that are soluble or non-ionic, useful in the invention, are those having a pH sufficient to control the pH desired for synthesizing a given molecular sieve, especially a SAPO molecular sieve. In a preferred embodiment, the polymeric base is soluble or the polymeric base is non-ionic, preferably the polymeric base is a non-ionic and soluble polymeric base, and most preferably the polymeric base is a polymeric imine. In one embodiment, the polymeric base of the invention has a pH in an aqueous solution, preferably water, from greater than 7 to about 14, more preferably from about 8 to about 14, most preferably from about 9 to 14.

In another embodiment, the non-volatile polymeric base is represented by the formula: (R—NH)ₓ, where (R—NH) is a polymeric or monomeric unit where R contains from 1 to 20 carbon atoms, preferably from 1 to 10 carbon atoms, more preferably from 1 to 6 carbon atoms, and most preferably from 1 to 4 carbon atoms; x is an integer from 1 to 500,000. In one embodiment, R is a linear, branched, or cyclic polymer, monomeric, chain, preferably a linear polymer chain having from 1 to 20 carbon atoms.

In another embodiment, the polymeric base is a water miscible polymeric base, preferably in an aqueous solution. In yet another embodiment, the polymeric base is a polyethylenimine that is represented by the following general formula: (—NHCH₂CH₂—)ₘ[—N(CH₂CH₂NH₂)CH₂CH₂—]ₙ), wherein m is from 10 to 20,000, and n is from 0 to 2,000, preferably from 1 to 2000.

In another embodiment, the polymeric base of the invention has a average molecular weight from about 500 to about 1,000,000, preferably from about 2,000 to about 800,000, more preferably from about 10,000 to about 750,000, and most preferably from about 50,000 to about 750,000.

In another embodiment, the mole ratio of the monomeric unit of the polymeric base of the invention, containing one basic group, to the templating agent(s) is less than 20, preferably less than 12, more preferably less than 10, even more preferably less than 8, still even more preferably less than 5, and most preferably less than 4.

Non-limiting examples of polymer bases include: epichlorohydrin modified polyethylenimine, ethoxylated polyethylenimine, polypropylenimine diamine dendrimers (DAB-Am-n), poly(allylamine) [CH₂CH(CH₂NH₂)]ₙ, poly(1,2-dihydro-2,2,4-trimethylquinoline), and poly(dimethylamine-co-epichlorohydrin-co-ethylenediamine).

In another embodiment the invention is directed to a method for synthesizing a molecular sieve utilizing a templating agent, preferably an organic templating agent such as an organic amine, an ammonium salt and/or an ammonium hydroxide, in combination with a polymeric base such as polyethylenimine.

In a typical synthesis of the molecular sieve, the phosphorous-, aluminum-, and/or silicon-containing components are mixed, preferably while stirring and/or agitation and/or seeding with a crystalline material, optionally with an alkali metal, in a solvent such as water, and one or more templating agents and a polymeric base, to form a synthesis mixture that is then heated under crystallization conditions of pressure and temperature as described in U.S. Pat. Nos. 4,440,871, 4,861,743, 5,096,684, and 5,126,308, which are all herein fully incorporated by reference. The polymeric base is combined with the at least one templating agent, and one or more of the aluminum source, phosphorous source, and silicon source, in any order, for example, simultaneously with one or more of the sources, premixed with one or more of the sources and/or templating agent, after combining the sources and the templating agent, and the like.

Generally, the synthesis mixture described above is sealed in a vessel and heated, preferably under autogenous pressure, to a temperature in the range of from about 80° C. to about 250° C., preferably from about 100° C. to about 250° C., more preferably from about 125° C. to about 225° C., even more preferably from about 150° C. to about 180° C. In another embodiment, the hydrothermal crystallization temperature is less than 225° C., preferably less than 200° C. to about 80° C., and more preferably less than 195° C. to about 100° C.

In yet another embodiment, the crystallization temperature is increased gradually or stepwise during synthesis, preferably the crystallization temperature is maintained constant, for a period of time effective to form a crystalline product. The time required to form the crystalline product is typically from immediately up to several weeks, the duration of which is usually dependent on the temperature; the higher the temperature the shorter the duration. In one embodiment, the crystalline product is formed under heating from about 30 minutes to around 2 weeks, preferably from about 45 minutes to about 240 hours, and more preferably from about 1 hour to about 120 hours.

In one embodiment, the synthesis of a molecular sieve is aided by seeds from another or the same framework type molecular sieve.

The hydrothermal crystallization is carried out with or without agitation or stirring, for example stirring or tumbling. The stirring or agitation during the crystallization period may be continuous or intermittent, preferably continuous agitation. Typically, the crystalline molecular sieve product is formed, usually in a slurry state, and is recovered by any standard technique well known in the art, for example centrifugation or filtration. The isolated or separated crystalline product, in an embodiment, is washed, typically, using a liquid such as water, from one to many times. The washed crystalline product is then optionally dried, preferably in air.

One method for crystallization involves subjecting an aqueous reaction mixture containing an excess amount of a templating agent and polymeric base, subjecting the mixture to crystallization under hydrothermal conditions, establishing an equilibrium between molecular sieve formation and dissolution, and then, removing some of the excess templating agent and/or organic base to inhibit dissolution of the molecular sieve. See for example U.S. Pat. No. 5,296,208, which is herein fully incorporated by reference.

Another method of crystallization is directed to not stirring a reaction mixture, for example a reaction mixture containing at a minimum, a silicon-, an aluminum-, and/or a phosphorous-composition, with a templating agent and a polymeric base, for a period of time during crystallization. See PCT WO 01/47810 published Jul. 5, 2001, which is herein fully incorporated by reference.

Other methods for synthesizing molecular sieves or modifying molecular sieves are described in U.S. Pat. No. 5,879,655 (controlling the ratio of the templating agent to phosphorous), U.S. Pat. No. 6,005,155 (use of a modifier without a salt), U.S. Pat. No. 5,475,182 (acid extraction), U.S. Pat. No. 5,962,762 (treatment with transition metal), U.S. Pat. Nos. 5,925,586 and 6,153,552 (phosphorous modified), U.S. Pat. No. 5,925,800 (monolith supported), U.S. Pat. No. 5,932,512 (fluorine treated), U.S. Pat. No. 6,046,373 (electromagnetic wave treated or modified), U.S. Pat. No. 6,051,746 (polynuclear aromatic modifier), U.S. Pat. No. 6,225,254 (heating template), PCT WO 01/36329 published May 25, 2001 (surfactant synthesis), PCT WO 01/25151 published Apr. 12, 2001 (staged acid addition), PCT WO 01/60746 published Aug. 23, 2001 (silicon oil), U.S. patent application Ser. No. 09/929,949 filed Aug. 15, 2001 (cooling molecular sieve), U.S. patent application Ser. No. 09/615,526 filed Jul. 13, 2000 (metal impregnation including copper), U.S. patent application Ser. No. 09/672,469 filed Sep. 28, 2000 (conductive microfilter), and U.S. patent application Ser. No. 09/754,812 filed Jan. 4, 2001 (freeze drying the molecular sieve), which are all herein fully incorporated by reference.

In one preferred embodiment, when a templating agent is used in the synthesis of a molecular sieve, it is preferred that the templating agent is substantially, preferably completely, removed after crystallization by numerous well known techniques, for example, heat treatments such as calcination. Calcination involves contacting the molecular sieve containing the templating agent with a gas, preferably containing oxygen, at any desired concentration at an elevated temperature sufficient to either partially or completely decompose and oxidize the templating agent.

Molecular sieves have either a high silicon (Si) to aluminum (Al) ratio or a low silicon to aluminum ratio, however, a low Si/Al ratio is preferred for SAPO synthesis. In one embodiment, the molecular sieve has a Si/Al ratio less than 0.65, preferably less than 0.40, more preferably less than 0.32, and most preferably less than 0.20. In another embodiment the molecular sieve has a Si/Al ratio in the range of from about 0.65 to about 0.10, preferably from about 0.40 to about 0.10, more preferably from about 0.32 to about 0.10, and more preferably from about 0.32 to about 0.15.

The pH of a reaction mixture containing at a minimum a silicon-, aluminum-, and/or phosphorous-composition, a templating agent, and a polymeric base should be in the range of from 2 to 10, preferably in the range of from 4 to 9, and most preferably in the range of from 5 to 8. The pH can be controlled by the addition of basic or acidic compounds as necessary to maintain the pH during the synthesis in the preferred range of from 4 to 9. In another embodiment, the templating agent and/or polymeric base is added to the reaction mixture of the silicon source and phosphorous source such that the pH of the reaction mixture does not exceed 10.

In one embodiment, the molecular sieves of the invention are combined with one or more other molecular sieves. In another embodiment, the preferred silicoaluminophosphate or aluminophosphate molecular sieves, or a combination thereof, are combined with one more of the following non-limiting examples of molecular sieves described in the following: Beta (U.S. Pat. No. 3,308,069), ZSM-5 (U.S. Pat. Nos. 3,702,886, 4,797,267 and 5,783,321), ZSM-11 (U.S. Pat. No. 3,709,979), ZSM-12 (U.S. Pat. No. 3,832,449), ZSM-12 and ZSM-38 (U.S. Pat. No. 3,948,758), ZSM-22 (U.S. Pat. No. 5,336,478), ZSM-23 (U.S. Pat. No. 4,076,842), ZSM-34 (U.S. Pat. No. 4,086,186), ZSM-35 (U.S. Pat. No. 4,016,245, ZSM-48 (U.S. Pat. No. 4,397,827), ZSM-58 (U.S. Pat. No. 4,698,217), MCM-1 (U.S. Pat. No. 4,639,358), MCM-2 (U.S. Pat. No. 4,673,559), MCM-3 (U.S. Pat. No. 4,632,811), MCM-4 (U.S. Pat. No. 4,664,897), MCM-5 (U.S. Pat. No. 4,639,357), MCM-9 (U.S. Pat. No. 4,880,611), MCM-10 (U.S. Pat. No. 4,623,527), MCM-14 (U.S. Pat. No. 4,619,818), MCM-22 (U.S. Pat. No. 4,954,325), MCM-41 (U.S. Pat. No. 5,098,684), M-41S (U.S. Pat. No. 5,102,643), MCM-48 (U.S. Pat. No. 5,198,203), MCM-49 (U.S. Pat. No. 5,236,575), MCM-56 (U.S. Pat. No. 5,362,697), ALPO-11 (U.S. Pat. No. 4,310,440), titanium aluminosilicates (TASO), TASO-45 (EP-A-0 229,-295), boron silicates (U.S. Pat. No. 4,254,297), titanium aluminophosphates (TAPO) (U.S. Pat. No. 4,500,651), mixtures of ZSM-5 and ZSM-11 (U.S. Pat. No. 4,229,424), ECR-18 (U.S. Pat. No. 5,278,345), SAPO-34 bound ALPO-5 (U.S. Pat. No. 5,972,203), PCT WO 98/57743 published Dec. 23, 1988 (molecular sieve and Fischer-Tropsch), U.S. Pat. No. 6,300,535 (MFI-bound zeolites), and mesoporous molecular sieves (U.S. Pat. Nos. 6,284,696, 5,098,684, 5,102,643 and 5,108,725), which are all herein fully incorporated by reference.

Method for Making Molecular Sieve Catalyst Compositions

Once the molecular sieve is synthesized, depending on the requirements of the particular conversion process, the molecular sieve is then formulated into a molecular sieve catalyst composition, particularly for commercial use. The molecular sieves synthesized above are made or formulated into catalysts by combining the synthesized molecular sieves with a binder and/or a matrix material to form a molecular sieve catalyst composition or a formulated molecular sieve catalyst composition. This formulated molecular sieve catalyst composition is formed into useful shape and sized particles by well-known techniques such as spray drying, pelletizing, extrusion, and the like.

There are many different binders that are useful in forming the molecular sieve catalyst composition. Non-limiting examples of binders that are useful alone or in combination include various types of hydrated alumina, silicas, and/or other inorganic oxide sol. One preferred alumina containing sol is aluminum chlorhydrol. The inorganic oxide sol acts like glue binding the synthesized molecular sieves and other materials such as the matrix together, particularly after thermal treatment. Upon heating, the inorganic oxide sol, preferably having a low viscosity, is converted into an inorganic oxide matrix component. For example, an alumina sol will convert to an aluminum oxide matrix following heat treatment.

Aluminum chlorhydrol, a hydroxylated aluminum based sol containing a chloride counter ion, has the general formula of AlₘOₙ(OH)₀Clₚ·x(H₂O) wherein m is 1 to 20, n is 1 to 8,o is 5 to 40, p is 2 to 15, and x is 0 to 30. In one embodiment, the binder is Al₁₃O₄(OH)₂₄Cl₇·12(H₂O) as is described in G. M. Wolterman, et al., Stud. Surf. Sci. and Catal., 76, pages 105-144 (1993), which is herein incorporated by reference. In another embodiment, one or more binders are combined with one or more other non-limiting examples of alumina materials such as aluminum oxyhydroxide, γ-alumina, boehmite, diaspore, and transitional aluminas such as α-alumina, β-alumina, γ-alumina, δ-alumina, ε-alumina, κ-alumina, and ρ-alumina, aluminum trihydroxide, such as gibbsite, bayerite, nordstrandite, doyelite, and mixtures thereof.

In another embodiment, the binders are alumina sols, predominantly comprising aluminum oxide, optionally including some silicon. In yet another embodiment, the binders are peptized alumina made by treating alumina hydrates such as pseudoboehmite, with an acid, preferably an acid that does not contain a halogen, to prepare sols or aluminum ion solutions. Non-limiting examples of commercially available colloidal alumina sols include Nalco 8676 available from Nalco Chemical Co., Naperville, Ill., and Nyacol available from The PQ Corporation, Valley Forge, Pa.

The molecular sieve synthesized above, in a preferred embodiment, is combined with one or more matrix material(s). Matrix materials are typically effective in reducing overall catalyst cost, act as thermal sinks assisting in shielding heat from the catalyst composition for example during regeneration, densifying the catalyst composition, increasing catalyst strength such as crush strength and attrition resistance, and to control the rate of conversion in a particular process.

Non-limiting examples of matrix materials include one or more of: rare earth metals, metal oxides including titania, zirconia, magnesia, thoria, beryllia, quartz, silica or sols, and mixtures thereof, for example silica-magnesia, silica-zirconia, silica-titania, silica-alumina and silica-alumina-thoria. In an embodiment, matrix materials are natural clays such as those from the families of montmorillonite and kaolin. These natural clays include subbentonites and those kaolins known as, for example, Dixie, McNamee, Georgia and Florida clays. Non-limiting examples of other matrix materials include: haloysite, kaolinite, dickite, nacrite, or anauxite. In one embodiment, the matrix material, preferably any of the clays, are subjected to well known modification processes such as calcination and/or acid treatment and/or chemical treatment.

In one preferred embodiment, the matrix material is a clay or a clay-type composition, preferably the clay or clay-type composition having a low iron or titania content, and most preferably the matrix material is kaolin. Kaolin has been found to form a pumpable, high solid content slurry, it has a low fresh surface area, and it packs together easily due to its platelet structure. A preferred average particle size of the matrix material, most preferably kaolin, is from about 0.1 μm to about 0.6 μm with a d₉₀ particle size distribution of less than about 1 μm.

In one embodiment, the binder, the molecular sieve and the matrix material are combined in the presence of a liquid to form a molecular sieve catalyst composition, where the amount of binder is from about 2% by weight to about 30% by weight, preferably from about 5% by weight to about 20% by weight, and more preferably from about 7% by weight to about 15% by weight, based on the total weight of the binder, the molecular sieve and matrix material, excluding the liquid (after calcination).

In another embodiment, the weight ratio of the binder to the matrix material used in the formation of the molecular sieve catalyst composition is from 0:1 to 1:15, preferably 1:15 to 1:5, more preferably 1:10 to 1:4, and most preferably 1:6 to 1:5. It has been found that a higher sieve content, lower matrix content, increases the molecular sieve catalyst composition performance, however, lower sieve content, higher matrix material, improves the attrition resistance of the composition.

Upon combining the molecular sieve and the matrix material, optionally with a binder, in a liquid to form a slurry, mixing, preferably rigorous mixing is needed to produce a substantially homogeneous mixture containing the molecular sieve. Non-limiting examples of suitable liquids include one or a combination of water, alcohol, ketones, aldehydes, and/or esters. The most preferred liquid is water. In one embodiment, the slurry is colloid-milled for a period of time sufficient to produce the desired slurry texture, sub-particle size, and/or sub-particle size distribution.

The molecular sieve and matrix material, and the optional binder, are in the same or different liquid, and are combined in any order, together, simultaneously, sequentially, or a combination thereof. In the preferred embodiment, the same liquid, preferably water is used. The molecular sieve, matrix material, and optional binder, are combined in a liquid as solids, substantially dry or in a dried form, or as slurries, together or separately. If solids are added together as dry or substantially dried solids, it is preferable to add a limited and/or controlled amount of liquid.

In one embodiment, the slurry of the molecular sieve, binder and matrix materials is mixed or milled to achieve a sufficiently uniform slurry of sub-particles of the molecular sieve catalyst composition that is then fed to a forming unit that produces the molecular sieve catalyst composition. In a preferred embodiment, the forming unit is spray dryer. Typically, the forming unit is maintained at a temperature sufficient to remove most of the liquid from the slurry, and from the resulting molecular sieve catalyst composition. The resulting catalyst composition when formed in this way takes the form of microspheres.

When a spray drier is used as the forming unit, typically, the slurry of the molecular sieve and matrix material, and optionally a binder, is co-fed to the spray drying volume with a drying gas with an average inlet temperature ranging from 200° C. to 550° C., and a combined outlet temperature ranging from 100° C. to about 225° C. In an embodiment, the average diameter of the spray dried formed catalyst composition is from about 40 μm to about 300 μm, preferably from about 50 μm to about 250 μm, more preferably from about 50 μm to about 200 μm, and most preferably from about 65 μm to about 90 μm.

During spray drying, the slurry is passed through a nozzle distributing the slurry into small droplets, resembling an aerosol spray into a drying chamber. Atomization is achieved by forcing the slurry through a single nozzle or multiple nozzles with a pressure drop in the range of from 100 psia to 1000 psia (690 kPaa to 6895 kPaa). In another embodiment, the slurry is co-fed through a single nozzle or multiple nozzles along with an atomization fluid such as air, steam, flue gas, or any other suitable gas.

In yet another embodiment, the slurry described above is directed to the perimeter of a spinning wheel that distributes the slurry into small droplets, the size of which is controlled by many factors including slurry viscosity, surface tension, flow rate, pressure, and temperature of the slurry, the shape and dimension of the nozzle(s), or the spinning rate of the wheel. These droplets are then dried in a co-current or counter-current flow of air passing through a spray drier to form a substantially dried or dried molecular sieve catalyst composition, more specifically a molecular sieve in powder form.

Generally, the size of the powder is controlled to some extent by the solids content of the slurry. However, control of the size of the catalyst composition and its spherical characteristics are controllable by varying the slurry feed properties and conditions of atomization.

Other methods for forming a molecular sieve catalyst composition are described in U.S. patent application Ser. No. 09/617,714 filed Jul. 17, 2000 (spray drying using a recycled molecular sieve catalyst composition), that is herein incorporated by reference.

In another embodiment, the formulated molecular sieve catalyst composition contains from about 1% to about 99%, more preferably from about 5% to about 90%, and most preferably from about 10% to about 80%, by weight of the molecular sieve based on the total weight of the molecular sieve catalyst composition.

In another embodiment, the weight percent of binder in or on the spray dried molecular sieve catalyst composition based on the total weight of the binder, molecular sieve, and matrix material is from about 2% by weight to about 30% by weight, preferably from about 5% by weight to about 20% by weight, and more preferably from about 7% by weight to about 15% by weight.

Once the molecular sieve catalyst composition is formed in a substantially dry or dried state, to further harden and/or activate the formed catalyst composition, a heat treatment such as calcination, at an elevated temperature is usually performed. A conventional calcination environment is air that typically includes a small amount of water vapor. Typical calcination temperatures are in the range from about 400° C. to about 1,000° C., preferably from about 500° C. to about 800° C., and most preferably from about 550° C. to about 700° C., preferably in a calcination environment such as air, nitrogen, helium, flue gas (combustion product lean in oxygen), or any combination thereof.

In one embodiment, calcination of the formulated molecular sieve catalyst composition is carried out in any number of well known devices including rotary calciners, fluid bed calciners, batch ovens, and the like. Calcination time is typically dependent on the degree of hardening of the molecular sieve catalyst composition and the temperature ranges from about 15 minutes to about 2 hours.

In a preferred embodiment, the molecular sieve catalyst composition is heated in nitrogen at a temperature of from about 600° C. to about 700° C. Heating is carried out for a period of time typically from 30 minutes to 15 hours, preferably from 1 hour to about 10 hours, more preferably from about 1 hour to about 5 hours, and most preferably from about 2 hours to about 4 hours.

Other methods for activating a molecular sieve catalyst composition, in particular where the molecular sieve is a reaction product of a combination of a silicon-, phosphorous-, and aluminum-sources, a templating agent, and a polymeric base, more particularly a silicoaluminophosphate catalyst composition (SAPO) are described in, for example, U.S. Pat. No. 5,185,310 (heating molecular sieve of gel alumina and water to 450° C.), PCT WO 00/75072 published Dec. 14, 2000 (heating to leave an amount of template), and U.S. application Ser. No. 09/558,774 filed Apr. 26, 2000 (rejuvenation of molecular sieve), which are all herein fully incorporated by reference.

The process for converting a feedstock, especially a feedstock containing one or more oxygenates, in the presence of a molecular sieve catalyst composition according to the invention, is carried out in a reaction process in a reactor, where the process is a fixed bed process, a fluidized bed process, preferably a continuous fluidized bed process, and most preferably a continuous high velocity fluidized bed process.

The reaction processes can take place in a variety of catalytic reactors such as hybrid reactors that have a dense bed or fixed bed zones and/or fast fluidized bed reaction zones coupled together, circulating fluidized bed reactors, riser reactors, and the like. Suitable conventional reactor types are described in for example U.S. Pat. No. 4,076,796, U.S. Pat. No. 6,287,522 (dual riser), and Fluidization Engineering, D. Kunil and O. Levenspiel, Robert E. Krieger Publishing Company, New York, N.Y. 1977, which are all herein fully incorporated by reference.

The preferred reactor types are riser reactors generally described in Riser Reactor, Fluidization and Fluid-Particle Systems, pages 48 to 59, F. A. Zenz and D. F. Othmer, Reinhold Publishing Corporation, New York, 1960, and U.S. Pat. No. 6,166,282 (fast-fluidized bed reactor), and U.S. patent application Ser. No. 09/564,613 filed May 4, 2000 (multiple riser reactor), which are all herein fully incorporated by reference.

In the preferred embodiment, a fluidized bed process or high velocity fluidized bed process includes a reactor system, a regeneration system and a recovery system.

The reactor system preferably is a fluid bed reactor system having a first reaction zone within one or more riser reactor(s) and a second reaction zone within at least one disengaging vessel, preferably comprising one or more cyclones. In one embodiment, the one or more riser reactor(s) and disengaging vessel is contained within a single reactor vessel. Fresh feedstock, preferably containing one or more oxygenates, optionally with one or more diluent(s), is fed to the one or more riser reactor(s) in which a zeolite or zeolite-type molecular sieve catalyst composition or coked version thereof is introduced. In one embodiment, the molecular sieve catalyst composition or coked version thereof is contacted with a liquid or gas, or combination thereof, prior to being introduced to the riser reactor(s), preferably the liquid is water or methanol, and the gas is an inert gas such as nitrogen.

In an embodiment, the amount of liquid feedstock fed separately or jointly with a vapor feedstock, to a reactor system is in the range of from 0.1 weight percent to about 85 weight percent, preferably from about 1 weight percent to about 75 weight percent, more preferably from about 5 weight percent to about 65 weight percent based on the total weight of the feedstock including any diluent contained therein. The liquid and vapor feedstocks are preferably of similar composition, or contain varying proportions of the same or different feedstock with the same or different diluent.

Oxygenates to Olefins Process

In a preferred embodiment of the process of the invention, the feedstock contains one or more oxygenates, more specifically, one or more organic compound(s) containing at least one oxygen atom. In the most preferred embodiment of the process of invention, the oxygenate in the feedstock is one or more alcohol(s), preferably aliphatic alcohol(s) where the aliphatic moiety of the alcohol(s) has from 1 to 20 carbon atoms, preferably from 1 to 10 carbon atoms, and most preferably from 1 to 4 carbon atoms. The alcohols useful as feedstock in the process of the invention include lower straight and branched chain aliphatic alcohols and their unsaturated counterparts.

Non-limiting examples of oxygenates include methanol, ethanol, n-propanol, isopropanol, methyl ethyl ether, dimethyl ether, diethyl ether, di-isopropyl ether, formaldehyde, dimethyl carbonate, dimethyl ketone, acetic acid, and mixtures thereof.

In the most preferred embodiment, the feedstock is selected from one or more of methanol, ethanol, dimethyl ether, diethyl ether or a combination thereof, more preferably methanol and dimethyl ether, and most preferably methanol.

The various feedstocks discussed above, particularly a feedstock containing an oxygenate, more particularly a feedstock containing an alcohol, are converted primarily into one or more olefin(s). The olefin(s) or olefin monomer(s) produced from the feedstock typically have from 2 to 30 carbon atoms, preferably 2 to 8 carbon atoms, more preferably 2 to 6 carbon atoms, still more preferably 2 to 4 carbons atoms, and most preferably ethylene and/or propylene.

Non-limiting examples of olefin monomer(s) include ethylene, propylene, butene-1, pentene-1,4-methyl-pentene-1, hexene-1, octene-1 and decene-1, preferably ethylene, propylene, butene-1, pentene-1,4-methyl-pentene-1, hexene-1, octene-1 and isomers thereof. Other olefin monomer(s) include unsaturated monomers, diolefins having 4 to 18 carbon atoms, conjugated or nonconjugated dienes, polyenes, vinyl monomers and cyclic olefins.

In the most preferred embodiment, the feedstock, preferably of one or more oxygenates, is converted in the presence of a molecular sieve catalyst composition into olefin(s) having 2 to 6 carbons atoms, preferably 2 to 4 carbon atoms. Most preferably, the olefin(s), alone or in combination, are converted from a feedstock containing an oxygenate, preferably an alcohol, most preferably methanol, to the preferred olefin(s) ethylene and/or propylene.

There are many processes used to convert feedstock into olefin(s) including various cracking processes such as steam cracking, thermal regenerative cracking, fluidized bed cracking, fluid catalytic cracking, deep catalytic cracking, and visbreaking.

The most preferred process is generally referred to as methanol-to-olefins (MTO). In a MTO process, typically an oxygenated feedstock, most preferably a methanol containing feedstock, is converted in the presence of a molecular sieve catalyst composition into one or more olefin(s), preferably and predominantly, ethylene and/or propylene, often referred to as light olefin(s).

In one embodiment of the process for conversion of a feedstock, preferably a feedstock containing one or more oxygenates, the amount of olefin(s) produced based on the total weight of hydrocarbon produced is greater than 50 weight percent, preferably greater than 60 weight percent, more preferably greater than 70 weight percent, and most preferably greater than 85 weight percent.

Increasing the selectivity of preferred hydrocarbon products such as ethylene and/or propylene from the conversion of an oxygenate using a molecular sieve catalyst composition is described in U.S. Pat. No. 6,137,022 (linear velocity), and PCT WO 00/74848 published Dec. 14, 2000 (methanol uptake index of at least 0.13), which are all herein fully incorporated by reference.

The feedstock, in one embodiment, contains one or more diluent(s), typically used to reduce the concentration of the feed stock, and are generally non-reactive to the feedstock or molecular sieve catalyst composition. Non-limiting examples of diluents include helium, argon, nitrogen, carbon monoxide, carbon dioxide, water, essentially non-reactive paraffins (especially alkanes such as methane, ethane, and propane), essentially non-reactive aromatic compounds, and mixtures thereof. The most preferred diluents are water and nitrogen, with water being particularly preferred.

The diluent, water, is used either in a liquid or a vapor form, or a combination thereof. The diluent is either added directly to a feedstock entering into a reactor or added directly into a reactor, or added with a molecular sieve catalyst composition. In one embodiment, the amount of diluent in the feedstock is in the range of from about 1 to about 99 mole percent based on the total number of moles of the feedstock and diluent, preferably from about 1 to 80 mole percent, more preferably from about 5 to about 50, most preferably from about 5 to about 25. In another embodiment, other hydrocarbons are added to a feedstock either directly or indirectly, and include olefin(s), paraffin(s), aromatic(s) (see for example U.S. Pat. No. 4,677,242, addition of aromatics) or mixtures thereof, preferably propylene, butylene, pentylene, and other hydrocarbons having 4 or more carbon atoms, or mixtures thereof.

The process for converting a feedstock, especially a feedstock containing one or more oxygenates, in the presence of a molecular sieve catalyst composition of the invention, is carried out in a reaction process in a reactor, where the process is a fixed bed process, a fluidized bed process, preferably a continuous fluidized bed process, and most preferably a continuous high velocity fluidized bed process.

The reaction processes can take place in a variety of catalytic reactors such as hybrid reactors that have a dense bed or fixed bed zones and/or fast fluidized bed reaction zones coupled together, circulating fluidized bed reactors, riser reactors, and the like. Suitable conventional reactor types are described in for example U.S. Pat. No. 4,076,796, U.S. Pat. No. 6,287,522 (dual riser), and Fluidization Engineering, D. Kunii and O. Levenspiel, Robert E. Krieger Publishing Company, New York, N.Y. 1977, which are all herein fully incorporated by reference.

The preferred reactor type are riser reactors generally described in Riser Reactor, Fluidization and Fluid-Particle Systems, pages 48 to 59, F. A. Zenz and D. F. Othmer, Reinhold Publishing Corporation, New York, 1960, and U.S. Pat. No. 6,166,282 (fast-fluidized bed reactor), and U.S. patent application Ser. No. 09/564,613 filed May 4, 2000 (multiple riser reactor), which are all herein fully incorporated by reference.

In the preferred embodiment, a fluidized bed process or high velocity fluidized bed process includes a reactor system, a regeneration system and a recovery system.

The reactor system preferably is a fluid bed reactor system having a first reaction zone within one or more riser reactor(s) and a second reaction zone within at least one disengaging vessel, preferably comprising one or more cyclones. In one embodiment, the one or more riser reactor(s) and disengaging vessel are contained within a single reactor vessel. Fresh feedstock, preferably containing one or more oxygenates, optionally with one or more diluent(s), is fed to the one or more riser reactor(s) in which a zeolite or zeolite-type molecular sieve catalyst composition or coked version thereof is introduced. In one embodiment, the molecular sieve catalyst composition or coked version thereof is contacted with a liquid or gas, or combination thereof, prior to being introduced to the riser reactor(s), preferably the liquid is water or methanol, and the gas is an inert gas such as nitrogen.

In an embodiment, the amount of liquid feedstock, is fed separately or jointly with a vapor feedstock, to a reactor system in the range of from about 0 weight percent to about 85 weight percent, preferably from about 1 weight percent to about 75 weight percent, more preferably from about 5 weight percent to about 65 weight percent, say, from about 0 weight percent to about 10 weight percent, based on the total weight of the feedstock including any diluent contained therein. The liquid and vapor feedstocks are preferably of similar composition, or contain varying proportions of the same or different feedstock with the same or different diluent.

Oxygenate-containing feedstock can be treated prior to its introduction to the oxygenates to olefins conversion reactor to remove non-volatile contaminants.

The feedstock entering the reactor system is preferably converted, partially or fully, in the first reactor zone into a gaseous effluent that enters the disengaging vessel along with a coked molecular sieve catalyst composition. In the preferred embodiment, cyclone(s) within the disengaging vessel are designed to separate the molecular sieve catalyst composition, preferably a coked molecular sieve catalyst composition, from the gaseous effluent containing one or more olefin(s) within the disengaging zone. Cyclones are preferred; however, gravity effects within the disengaging vessel will also separate the catalyst compositions from the gaseous effluent. Other methods for separating the catalyst compositions from the gaseous effluent include the use of plates, caps, elbows, and the like.

In one embodiment of the disengaging system, the disengaging system includes a disengaging vessel. Typically, a lower portion of the disengaging vessel is a stripping zone. In the stripping zone the coked molecular sieve catalyst composition is contacted with a gas, preferably one or a combination of steam, methane, carbon dioxide, carbon monoxide, hydrogen, or an inert gas such as argon, preferably steam, to recover adsorbed hydrocarbons from the coked molecular sieve catalyst composition that is then introduced to the regeneration system. In another embodiment, the stripping zone is in a separate vessel from the disengaging vessel and the gas is passed at a gas hourly superficial velocity (GHSV) of from 1 hr⁻¹ to about 20,000 hr⁻¹ based on the volume of gas to volume of coked molecular sieve catalyst composition, preferably at an elevated temperature from 250° C. to about 750° C., preferably from about 350° C. to 650° C., over the coked molecular sieve catalyst composition.

The conversion temperature employed in the conversion process, specifically within the reactor system, is in the range of from about 200° C. to about 1000° C., preferably from about 250° C. to about 800° C., more preferably from about 250° C. to about 750° C., yet more preferably from about 300° C. to about 650° C., yet even more preferably from about 350° C. to about 600° C., most preferably from about 350° C. to about 550° C.

The conversion pressure employed in the conversion process, specifically within the reactor system, varies over a wide range including autogenous pressure. The conversion pressure is based on the partial pressure of the feedstock exclusive of any diluent therein. Typically the conversion pressure employed in the process is in the range of from about 0.1 kPaa to about 5 MPaa, preferably from about 5 kPaa to about 1 MPaa, and most preferably from about 20 kPaa to about 500 kPaa.

The weight hourly space velocity (WHSV), particularly in a process for converting a feedstock containing one or more oxygenates in the presence of a molecular sieve catalyst composition within a reaction zone, is defined as the total weight of the feedstock excluding any diluents to the reaction zone per hour per weight of molecular sieve in the molecular sieve catalyst composition in the reaction zone. The WHSV is maintained at a level sufficient to keep the catalyst composition in a fluidized state within a reactor.

Typically, the WHSV ranges from about 1 hr⁻¹ to about 5000 hr⁻¹, preferably from about 2 hr⁻¹ to about 3000 hr⁻¹, more preferably from about 5 hr⁻¹ to about 1500 hr⁻¹, and most preferably from about 10 hr⁻¹ to about 1000 hr⁻¹. In one preferred embodiment, the WHSV is greater than 20 hr⁻¹, preferably the WHSV for conversion of a feedstock containing methanol and dimethyl ether is in the range of from about 20 hr⁻¹ to about 300 hr⁻¹.

The superficial gas velocity (SGV) of the feedstock including diluent and reaction products within the reactor system is preferably sufficient to fluidize the molecular sieve catalyst composition within a reaction zone in the reactor. The SGV in the process, particularly within the reactor system, more particularly within the riser reactor(s), is at least 0.1 meter per second (m/sec), preferably greater than 0.5 m/sec, more preferably greater than 1 m/sec, even more preferably greater than 2 m/sec, yet even more preferably greater than 3 m/sec, and most preferably greater than 4 m/sec, e.g., greater than about 15 m/sec. See, for example, U.S. patent application Ser. No. 09/708,753 filed Nov. 8, 2000, which is herein incorporated by reference.

In one preferred embodiment of the process for converting an oxygenates to olefin(s) using a silicoaluminophosphate molecular sieve catalyst composition, the process is operated at a WHSV of at least 20 hr⁻¹ and a Temperature Corrected Normalized Methane Selectivity (TCNMS) of less than 0.016, preferably less than or equal to 0.01. See for example U.S. Pat. No. 5,952,538, which is herein fully incorporated by reference.

In another embodiment of the process for converting an oxygenate such as methanol to one or more olefin(s) using a molecular sieve catalyst composition, the WHSV is from 0.01 hr⁻¹ to about 100 hr⁻¹, at a temperature of from about 350° C. to 550° C., and silica to Me₂O₃ (Me is selected from Group 13 (IIIA), Groups 8, 9 and 10 (VIII) elements) from the Periodic Table of Elements), and a molar ratio of from 300 to 2500. See, for example, EP-0 642 485 B1, which is herein fully incorporated by reference.

Other processes for converting an oxygenate such as methanol to one or more olefin(s) using a molecular sieve catalyst composition are described in PCT WO 01/23500 published Apr. 5, 2001 (propane reduction at an average catalyst feedstock exposure of at least 1.0), which is herein incorporated by reference.

The coked molecular sieve catalyst composition is withdrawn from the disengaging vessel, preferably by one or more cyclones(s), and introduced to the regeneration system. The regeneration system comprises a regenerator where the coked catalyst composition is contacted with a regeneration medium, preferably a gas containing oxygen, under general regeneration conditions of temperature, pressure and residence time.

Non-limiting examples of the regeneration medium include one or more of oxygen, O₃, SO₃, N₂O, NO, NO₂, N₂O₅, air, air diluted with nitrogen or carbon dioxide, oxygen and water (U.S. Pat. No. 6,245,703), carbon monoxide and/or hydrogen. The regeneration conditions are those capable of burning coke from the coked catalyst composition, preferably to a level less than 0.5 weight percent based on the total weight of the coked molecular sieve catalyst composition entering the regeneration system. The coked molecular sieve catalyst composition withdrawn from the regenerator forms a regenerated molecular sieve catalyst composition.

The regeneration temperature is in the range of from about 200° C. to about 1500° C., preferably from about 300° C. to about 1000° C., more preferably from about 450° C. to about 750° C., and most preferably from about 550° C. to 700° C. The regeneration is in the range of from about 10 psia (68 kPaa) to about 500 psia (3448 kPaa), preferably from about 15 psia (103 kPaa) to about 250 psia (1724 kPaa), and more preferably from about 20 psia (138 kPaa) to about 150 psia (1034 kPaa). Typically, the pressure is less than about 60 psia (414 kPaa).

The preferred residence time of the molecular sieve catalyst composition in the regenerator is in the range of from about one minute to several hours, most preferably about one minute to 100 minutes, and the preferred volume of oxygen in the flue gas is in the range of from about 0.01 mole percent to about 5 mole percent based on the total volume of the gas.

In one embodiment, regeneration promoters, typically metal containing compounds such as platinum, palladium and the like, are added to the regenerator directly, or indirectly, for example with the coked catalyst composition. Also, in another embodiment, a fresh molecular sieve catalyst composition is added to the regenerator containing a regeneration medium of oxygen and water as described in U.S. Pat. No. 6,245,703, which is herein fully incorporated by reference.

In an embodiment, a portion of the coked molecular sieve catalyst composition from the regenerator is returned directly to the one or more riser reactor(s), or indirectly, by pre-contacting with the feedstock, or contacting with fresh molecular sieve catalyst composition, or contacting with a regenerated molecular sieve catalyst composition or a cooled regenerated molecular sieve catalyst composition described below.

The burning of coke is an exothermic reaction, and in an embodiment, the temperature within the regeneration system is controlled by various techniques in the art including feeding a cooled gas to the regenerator vessel, operated either in a batch, continuous, or semi-continuous mode, or a combination thereof. A preferred technique involves withdrawing the regenerated molecular sieve catalyst composition from the regeneration system and passing the regenerated molecular sieve catalyst composition through a catalyst cooler that forms a cooled regenerated molecular sieve catalyst composition. The catalyst cooler, in an embodiment, is a heat exchanger that is located either internal or external to the regeneration system.

In one embodiment, the cooler regenerated molecular sieve catalyst composition is returned to the regenerator in a continuous cycle, alternatively, (see U.S. patent application Ser. No. 09/587,766 filed Jun. 6, 2000) a portion of the cooled regenerated molecular sieve catalyst composition is returned to the regenerator vessel in a continuous cycle, and another portion of the cooled molecular sieve regenerated molecular sieve catalyst composition is returned to the riser reactor(s), directly or indirectly, or a portion of the regenerated molecular sieve catalyst composition or cooled regenerated molecular sieve catalyst composition is contacted with by-products within the gaseous effluent (PCT WO 00/49106 published Aug. 24, 2000), which are all herein fully incorporated by reference. In another embodiment, a regenerated molecular sieve catalyst composition contacted with an alcohol, preferably ethanol, 1-propanol, 1-butanol or mixture thereof, is introduced to the reactor system, as described in U.S. patent application Ser. No. 09/785,122 filed Feb. 16, 2001, which is herein fully incorporated by reference.

Other methods for operating a regeneration system are disclosed in U.S. Pat. No. 6,290,916 (controlling moisture), which is herein fully incorporated by reference.

The regenerated molecular sieve catalyst composition withdrawn from the regeneration system, preferably from the catalyst cooler, is combined with a fresh molecular sieve catalyst composition and/or re-circulated molecular sieve catalyst composition and/or feedstock and/or fresh gas or liquids, and returned to the riser reactor(s). In another embodiment, the regenerated molecular sieve catalyst composition withdrawn from the regeneration system is returned to the riser reactor(s) directly, optionally after passing through a catalyst cooler. In one embodiment, a carrier, such as an inert gas, feedstock vapor, steam or the like, semi-continuously or continuously, facilitates the introduction of the regenerated molecular sieve catalyst composition to the reactor system, preferably to the one or more riser reactor(s).

In one embodiment, the optimum level of coke on the molecular sieve catalyst composition in the reaction zone is maintained by controlling the flow of the regenerated molecular sieve catalyst composition or cooled regenerated molecular sieve catalyst composition from the regeneration system to the reactor system. There are many techniques for controlling the flow of a molecular sieve catalyst composition described in Michael Louge, Experimental Techniques, Circulating Fluidized Beds, Grace, Avidan and Knowlton, eds., Blackie, 1997 (336-337), which is herein incorporated by reference. This is referred to as the complete regeneration mode. In another embodiment, referred to as the partial regeneration mode, the optimum level of coke on the molecular sieve catalyst composition in the reaction zone is maintained by controlling the flow rate of the oxygen-containing gas flow to the regenerator.

Coke levels on the molecular sieve catalyst composition are measured by withdrawing from the conversion process the molecular sieve catalyst composition at a point in the process and determining its carbon content. Typical levels of coke on the molecular sieve catalyst composition after regeneration are less than about 15 weight percent, say, less than about 2 weight percent, with levels of coke ranging from about 0.01 weight percent to about 15 weight percent, preferably from about 0.05 weight percent to about 10 weight percent, based on the total weight of the molecular sieve and not the total weight of the molecular sieve catalyst composition.

In one embodiment, the molecular sieve catalyst composition in the reaction zone contains in the range of from about 1 to 50 weight percent, preferably from about 2 to 30 weight percent, more preferably from about 2 to about 20 weight percent, and most preferably from about 2 to about 10 weight percent coke or carbonaceous deposit based on the total weight of the mixture of molecular sieve catalyst compositions. See, for example, U.S. Pat. No. 6,023,005, which is herein fully incorporated by reference. It is recognized that the molecular sieve catalyst composition in the reaction zone is made up of a mixture of regenerated catalyst and catalyst that has ranging levels of carbonaceous deposits. The measured level of carbonaceous deposits thus represents an average of the levels for an individual catalyst particle.

The present invention solves the current needs in the art by providing a method for converting a feed including an oxygenate to a product including a light olefin. The method of the present invention is conducted in a reactor apparatus. As used herein, the term “reactor apparatus” refers to an apparatus which includes at least a place in which an oxygenates to olefins conversion reaction takes place. As further used herein, the term “reaction zone” refers to the portion of a reactor apparatus in which the oxygenates to olefins conversion reaction takes place and is used synonymously with the term “reactor.” Desirably, the reactor apparatus includes a reaction zone, an inlet zone and a disengaging zone. The “inlet zone” is the portion of the reactor apparatus into which feed and catalyst are introduced. The “reaction zone” is the portion of the reactor apparatus in which the feed is contacted with the catalyst under conditions effective to convert the oxygenate portion of the feed into a light olefin product. The “disengaging zone” is the portion of the reactor apparatus in which the catalyst and any additional solids in the reactor are separated from the products. Typically, the reaction zone is positioned between the inlet zone and the disengaging zone.

A preferred embodiment of a reactor system for the present invention is a circulating fluid bed reactor with continuous regeneration, similar to a modern fluid catalytic cracker. Fixed beds are not practical for the process because oxygenates to olefins conversion is a highly exothermic process which requires several stages with intercoolers or other cooling devices. The reaction also results in a high pressure drop due to the production of low pressure, low density gas.

Because the catalyst must be regenerated frequently, the reactor should allow easy removal of a portion of the catalyst to a regenerator, where the catalyst is subjected to a regeneration medium, preferably a gas comprising oxygen, most preferably air, to burn off coke from the catalyst, which restores the catalyst activity. The conditions of temperature, oxygen partial pressure, and residence time in the regenerator should be selected to achieve a coke content on regenerated catalyst of no greater than 10 carbon atoms per acid site of the molecular sieve in the catalyst, or the formulated catalyst itself. At least a portion of the regenerated catalyst should be returned to the reactor.

Recovery System

The gaseous effluent is withdrawn from the disengaging zone of the reactor apparatus and is passed through a recovery system. There are many well-known recovery systems, techniques and sequences that are useful in separating olefin(s) and purifying olefin(s) from the gaseous effluent. Recovery systems generally comprise one or more or a combination of various separation, fractionation and/or distillation towers, columns, splitters, or trains, for reaction systems such as ethylbenzene manufacture (see, U.S. Pat. No. 5,476,978, fully incorporated herein-by reference) and other derivative processes such as aldehydes, ketones and ester manufacture (see U.S. Pat. No. 5,675,041, fully incorporated herein by reference), and other associated equipment for example various condensers, beat exchangers, refrigeration systems or chill trains, compressors, knock-out drums or pots, pumps, and the like.

Non-limiting examples of these towers, columns, splitters or trains used alone or in combination include one or more of a demethanizer, preferably a high temperature demethanizer, a deethanizer, a depropanizer, a wash tower often referred to as a caustic wash tower and/or quench tower, absorbers, adsorbers, membranes, demethanizer, deethanizer, deetherizer, C₂ splitter, depropanizer, C₃ splitter, debutanizer, and the like.

Various recovery systems useful for recovering predominately olefin(s), preferably prime or light olefin(s) such as ethylene, propylene and/or butene are described in U.S. Pat. No. 5,960,643 (secondary rich ethylene stream), U.S. Pat. Nos. 5,019,143, 5,452,581 and 5,082,481 (membrane separations), U.S. Pat. No. 5,672,197 (pressure dependent adsorbents), U.S. Pat. No. 6,069,288 (hydrogen removal), U.S. Pat. No. 5,904,880 (recovered methanol to hydrogen and carbon dioxide in one step), U.S. Pat. No. 5,927,063 (recovered methanol to gas turbine power plant), and U.S. Pat. No. 6,121,504 (direct product quench), U.S. Pat. No. 6,121,503 (high purity olefins without superfractionation), and U.S. Pat. No. 6,293,998 (pressure swing adsorption), which are all herein fully incorporated by reference.

Generally accompanying most recovery systems is the production, generation or accumulation of additional products, by-products and/or contaminants along with the preferred prime products. The preferred prime products, the light olefins, such as ethylene and propylene, are typically purified for use in derivative manufacturing processes such as polymerization processes. Therefore, in the most preferred embodiment of the recovery system, the recovery system also includes a purification system. For example, the light olefin(s) produced particularly in a MTO process are passed through a purification system that removes low levels of by-products or contaminants.

Non-limiting examples of contaminants and by-products include generally polar compounds such as water, alcohols, carboxylic acids, ethers, carbon oxides, sulfur compounds such as hydrogen sulfide, carbonyl sulfides and mercaptans, ammonia and other nitrogen compounds, arsine, phosphine and chlorides. Other contaminants or by-products include hydrogen and hydrocarbons such as acetylene, methyl acetylene, propadiene, butadiene and butyne.

Other recovery systems that include purification systems, for example for the purification of olefin(s), are described in Kirk-Othmer Encyclopedia of Chemical Technology, 4th Edition, Volume 9, John Wiley & Sons, 1996, pages 249-271 and 894-899, which is herein incorporated by reference. Purification systems are also described in for example, U.S. Pat. No. 6,271,428 (purification of a diolefin hydrocarbon stream), U.S. Pat. No. 6,293,999 (separating propylene from propane), and U.S. patent application Ser. No. 09/689,363 filed Oct. 20, 2000 (purge stream using hydrating catalyst), which are herein incorporated by reference.

Hydrogenation Reactor

The present invention especially relates to hydrogenating acetylene, methyl acetylene, and/or propadiene (allene) in oxygenates to olefins (OTO) product streams. These highly unsaturated contaminants can be removed from OTO product streams by selective hydrogenation in the OTO recovery system, typically by front-end hydrogenation in at least one hydrogenation reactor or converter situated between a compression stage located downstream from the oxygenates to olefins reactor outlet, and a cryogenic fractionation stage, preferably the cryogenic fractionation stage located furthest upstream, utilizing a suitable refrigerant as known to those skilled in the art, to effect fractionation. In one embodiment, a single hydrogenation reactor is utilized, located between a compression stage and a cryogenic fractionation stage

Acetylene has the empirical formula C₂H₂, with a triple bond between the two carbon atoms in the molecule. By selectively adding hydrogen to acetylene, the desirable mono-olefin ethylene, having the empirical formula C₂H₄ is produced. Methyl acetylene and propadiene both have the empirical formula C₃H₄ and are collectively referred to as MAPD. Methyl acetylene has a triple bond between two of its three carbon atoms, while propadiene has two double bonds between its three carbon atoms. By selectively adding hydrogen to methyl acetylene and propadiene, the olefin propylene, having the empirical formula C₃H₆ is produced. Propylene is another desirable product in the OTO process. Acetylene, methyl acetylene, and propadiene are more highly unsaturated than the desired mono-olefin products from the OTO process, which possess but a single carbon-to-carbon double bond.

The reactivity of highly unsaturated acetylene and MAPD in the presence of a hydrogenating catalyst is typically higher than the activity of mono-olefin compounds. This increased activity allows for the selective hydrogenation of the highly unsaturated compounds in a stream of mono-olefin compounds. However, since the concentration of the mono-olefin compounds in the reactor effluent is many times higher than the concentration of the more highly unsaturated compounds, some of the mono-olefin compounds will nonetheless hydrogenate. Minimizing this undesirable reaction is a major goal of catalyst selection and the selection of proper reaction conditions.

Acetylene and MAPD occur in very low concentrations in oxygenates to olefins reactor effluent as compared to steam cracker effluent. In steam cracking from about 1 to about 3 percent of the effluent from the steam cracker is acetylene or MAPD. In comparison, a typical OTO process produces less than 0.01 wt % MAPD and less than 0.01 wt % acetylene. Typical manufacturing specifications for ethylene require that less than 0.5 mole ppm acetylene exists in the final product, while typical manufacturing specifications for propylene require that less than 2.9 mole ppm MAPD exist in the final product. Reaching and achieving these manufacturing specifications using front-end hydrogenation processes calls for obtaining even lower concentrations of acetylene and MAPD following the hydrogenation processes, because downstream separations can concentrate these compounds within a single product stream. For example, in an OTO product stream comprising both ethylene and propylene most of the acetylene left in the product stream will eventually comprise part of the ethylene product and most of the MAPD will comprise part of the propylene product stream.

Starting with an OTO product stream which has very small concentrations of these highly saturated compounds allows for a much less demanding hydrogenation process than the process used for a steam cracking stream to achieve and surpass the manufacturing specifications. The less rigorous hydrogenation requirement allows for using a front-end hydrogenation procedure without excessive hydrogenation of olefin products.

Hydrogenation Catalyst

The primary catalyst type used to hydrogenate acetylene and MAPD is a transition metal supported on alumina. In an embodiment, the hydrogenation catalyst comprises a metal selected from the group consisting of Ni, Pd and Pt, typically Pd. The hydrogenation catalyst can further comprise a metal selected from the group consisting of Cu, Ag and Au. The hydrogenation catalyst typically comprises an inorganic oxide support, e.g., alumina, silica and/or silica-alumina.

The most common metals are nickel, palladium, platinum and silver. A preferable catalyst is a palladium-based catalyst on an alumina support. Palladium-based catalysts are well-suited to balance activity (how fast the acetylene and MAPD compounds are hydrogenated) with selectivity (how much acetylene and MAPD is hydrogenated in comparison to other hydrocarbons, for example the olefin products). In still another embodiment, the hydrogenation catalyst comprises palladium and silver, supported on calcium carbonate. A typical palladium/alumina catalyst is formed into pellets of cylindrical shape having a diameter of about 3 mm and a height of 3 mm.

Suitable catalysts for the present invention have a hydrogenation metal loading ranging from about 0.001 to about 2 wt %, say, from about 0.01 to about 1 wt %. Commercially available catalysts suitable for use in the present invention hydrogenation reactor include G83C and G58 available from Sud Chemie, of Munich, Germany, as well as E-Series catalysts available from Chevron-Phillips of The Woodland, Texas. The hydrogenation catalyst can be used in a variety of known reactors including fixed-bed and fluidized bed reactors. In another embodiment, the hydrogenation catalyst comprises from about 0.001 to about 2 wt % of the hydrogenation metal, say, from about 0.01 to about 1 wt % palladium.

The selective hydrogenation process can be carried out at a variety of conditions. The temperature can begin at a low temperature assuring that very little mono-olefin product is hydrogenated during the selective hydrogenation process. As the hydrogenation catalyst ages, its activity typically decreases due to a buildup of carbon deposits. The reaction temperature can be raised to compensate for this decrease in reaction rate. However, the reaction temperature should not be raised so high that the hydrogenation of olefin compounds begins to rapidly occur. Thus temperature must be controlled during the reaction process, inasmuch as the hydrogenation of highly unsaturated hydrocarbons is a strongly exothermic process.

For the hydrogenation of acetylene, MA, and/or PD in a mixture of olefins including ethylene and propylene, suitable reaction temperatures (as measured by the temperature of the feed at the hydrogenation reactor inlet) range from about 110° to about 250° F. (from about 43° C. to about 121° C.), say, from about 160° to about 210° F. (from about 71° C. to about 99° C.). The hydrogenation reactor is operated at conditions comprising from about 9000 to about 25000 weight hourly space velocity, say, from about 10000 to about 18000 weight hourly space velocity, and from about 150 to about 500 psig (1140 to about 3550 kPaa), say, from about 250 to about 450 psig (from about 1830 kPaa to about 3210 kPaa).

Hydrogenation of the mono-olefins in the effluent stream is also prevented by excess carbon monoxide in the effluent stream. The excess carbon monoxide is preferably absorbed on hydrogenation catalysts, e.g., palladium-based catalysts. The absorbed carbon monoxide blocks absorption of mono-olefins onto the palladium catalyst, while still enabling the absorption of highly saturated hydrocarbons such as acetylene and MAPD.

The feed directed to the inlet of the hydrogenation reactor is typically a C₃₋ overhead stream comprising from about 100 ppm to about 2000 ppm CO, say, from about 200 ppm to about 400 ppm CO, from about 0.1 ppm to about 40 ppm acetylene, say, from about 0.1 ppm to about 10 ppm acetylene, from about 0 ppm to about 80 ppm propadiene, say, from about 0 ppm to about 40 ppm propadiene, and from about 0 to about 80 ppm methyl acetylene, say, from about 0 to about 40 ppm methyl acetylene. The stream directed to the hydrogenation reactor inlet has a molar ratio of carbon monoxide/acetylene ranging from about 100 to about 20, say, from about 80 to about 40.

The less rigorous hydrogenation requirement for an OTO effluent stream also allows for a less active and more selective catalyst to be used for the hydrogenation process, than that used in treating steam cracker effluent. In addition, lower temperatures can be used during the hydrogenation process, decreasing the rate of acetylene and MAPD hydrogenation, but also decreasing the rate and amount of olefin products that are hydrogenated. Additionally, the hydrogenation catalyst can be used for a longer period of time before reaching the temperature at which the hydrogenation of olefin compounds begins to occur rapidly.

The concentration of hydrogen in the effluent from the OTO process is in excess of the amount that is stoichiometrically required to hydrogenate all of the acetylene and MAPD in the effluent stream. However, the concentration of hydrogen in this stream is not so great that uncontrollable hydrogenation of the olefin products results during the hydrogenation process. Preferably, the molar concentration of hydrogen in the effluent stream is less than about 20% of the concentration of the olefin products, more preferably less than about 10%, most preferably less than about 5%.

A flow diagram is shown in the FIGURE which depicts an embodiment of the invention in which the hydrogenation of the OTO effluent stream occurs before splitting the stream into separate hydrocarbon product streams. In the FIGURE, a methanol-containing feed stream 10 is fed into oxygenates to olefins reactor 12. The oxygenates to olefins reactor 12 contains a SAPO-34-containing catalyst and is maintained at oxygenates to olefins conversion conditions sufficient to convert the methanol-containing feed stream 10 into an effluent stream 14 containing a variety of hydrocarbon and oxygenate compounds. Flue gas 13 is removed from the reactor 12. The gaseous effluent stream 14 is directed into a bottom portion of a quench tower 18 in which cooling water is directed into an upper portion of quench tower 18 at a rate sufficient to condense most of the water and unreacted oxygenate feed present in effluent stream 14. Quench tower 18 contains a suitable packing known to those skilled in the art that aids heat transfer and mixing of the gaseous effluent stream 14 and the cooling water. Stream 20, the bottoms from the quench tower 18, contains warmed quenching water, condensed water, absorbed oxygenates and condensed unreacted methanol from effluent stream 14. Stream 22, the overhead stream from quench tower 18, contains C₂ and higher olefins, e.g., C₂ to C₄ olefin and other hydrocarbon products, including acetylene, and optionally methyl acetylene and/or propadiene, hydrogen and oxygenates that were not completely absorbed by the water in the quench tower 18.

Stream 22 is saturated in water vapor and still contains unacceptable levels of oxygenated hydrocarbons. Even low levels, typically 1 ppm or less, of water and oxygenates can poison polyolefin catalysts if these contaminants are in the final olefin products used as polymerization feeds. Some of the water and oxygenates in stream 22 can be removed simply by compressing the stream. Compressing the stream condenses some of the water and oxygenates. Compressing the stream also minimizes the size and increases the effectiveness of downstream processes. Various washes and separations can be subsequently carried out to remove water and oxygenates from stream 22. Compression apparatus 24 provides at least a single stage compression, preferably a plural stage compression, e.g., a three stage compression. The compression apparatus effluent 26 is directed to a separation apparatus 28 which comprises at least one fractionator column and which provides a C₃ overhead stream 30 comprising ethylene, propylene, hydrogen, CO and acetylene. Typically, the separation apparatus 28 comprises a means for treating the separation apparatus bottoms stream, e.g., at least partially removing water and unreacted oxygenates using a fractionation tower making a cut between propylene and propane, which is especially useful in effecting the separation of oxygenated hydrocarbons like dimethyl ether from acetylene, propadiene and methyl acetylene which have boiling points in the range of −46° to 15° C.

Apparatus 28 also includes a fractionation of all the condensed water with oxygenated hydrocarbons. This separation frees the water of sufficient levels of oxygenated hydrocarbons that is to be sent to other wastewater treatment facilities. The oxygenated hydrocarbons in stream 32 are returned to oxygenates to olefins reactor 12 and waste water 34 is removed.

The bottoms stream of the separation apparatus 28 may also be further treated to provide a C₅ product stream 36 and a C₄ product stream 38, e.g., by employing a depentanizer.

The C₃ overhead stream 30 is obtained by fractionating the dimethyl ether and heavier components out of stream 26. Stream 30 then becomes primarily component that includes some level of acetylene, propadiene and methyl acetylene. The fractionator so used has been referred to as a depropanizer, depropylenizer or as a deetherizer.

The C₃ overhead stream 30 contains propylene, ethylene, hydrogen, CO and acetylene, and optionally, propane, depending on the particular fractionation carried out to obtain stream 30. Stream 30 also may optionally contain methyl acetylene and/or propadiene, particularly where dimethyl ether has been substantially removed with the use of a deetherizer.

Stream 30, particularly where it contains acid components such as carbon dioxide or carbonic acid, can be directed to an optional caustic treater 40 to effect removal of acidic components, providing a caustic treated stream 42. Stream 30, or in the case of the optional caustic treater, stream 42, is directed to an optional molecular sieve dryer 44 which removes residual moisture and provides a dried stream 46. Stream(s) 30, 42 and/or stream 46, depending on the optional apparatus in service, is directed to the hydrogenation reactor 48.

The hydrogenation reactor 48 contains a fixed bed reactor containing a suitable hydrogenation catalyst, e.g., a palladium catalyst on an alumina support. Inasmuch as hydrogenation catalysts are sulfur-sensitive, it is well-known to those skilled in the art that care should be taken to provide a sulfur-free or low sulfur stream to the hydrgenation reactor. The hydrogenation reactor 48 is operated under mild hydrogenation conditions (as set out above). Sending a hydrocarbon stream which contains non-hydrogen-reacting hydrocarbons over the hydrogenation catalyst can help control reaction temperatures inasmuch as the non-reacting hydrocarbons can act as a heat sink for the exothermic reaction. Optional externally provided hydrogen 50 can be added to the hydrogenation reactor as needed. The hydrogenation reactor 48, produces a product stream 52 with acetylene and MAPD levels significantly below the levels specified for the olefin products. Product stream 52 can then be directed via an optional molecular sieve dryer 54 which provides a dried product stream 56 to a cryogenic recovery train apparatus 58 which provides a C₃⁼ product 60, a C₂⁼ product 62, a C₁ and H₂ tail gas 64 and a C₂ and C₃ fuel 66.

Apparatus 58 will include a deethanizer to separate the C₃⁼ product, stream 60 from the C₂₋ components. The C₂ components are chilled in order to facilitate the separation of C₁₋ components from the C₂₊ components. The C₁ and H₂, stream 64 will be the overhead product of the demethanizer. The C₂s are separated by another fractionation step which produces the C₂⁼ product, stream 62 and the C₂ which become part of the fuel stream 66.

The foregoing embodiment requires only a single hydrogenation step for conversion of alkynes derived from oxygenates to olefins conversion. The hydrogen reactor location and its operation minimize the need for externally provided hydrogen and eliminate the need for extra driers normally required for separate acetylene and MAPD hydrogenation reactors utilized for treating steam cracking effluent.

1. A method for removing acetylene from an olefinic stream, comprising:
fractionating said olefinic stream comprising C₂ to C₄ olefin, hydrogen and acetylene, in a fractionator to provide a C₃₋ overhead stream comprising ethylene, propylene, hydrogen, CO and acetylene;
directing said C₃₋ overhead stream to an inlet of a hydrogenation reactor and contacting said C₃₋ overhead stream with a hydrogenation catalyst under conditions sufficient to hydrogenate substantially all of said acetylene to olefin without substantially converting said ethylene and/or said propylene; and
removing a purified olefin stream from the hydrogenation reactor.

2. The method of claim 1 wherein said C₃₋ overhead stream directed to said hydrogenation reactor inlet has a temperature ranging from about 110° to about 250° F.

3. The method of claim 2 wherein said hydrogenation reactor is operated at conditions comprising from about 9000 to about 25000 weight hourly space velocity and from about 150 to about 500 psig.

4. The method of claim 2 wherein said C₃₋ overhead stream directed to said inlet comprises from about 100 ppm to about 2000 ppm CO, from about 0.1 ppm to about 40 ppm acetylene, from about 0 ppm to about 80 ppm propadiene, and from about 0 ppm to about 80 ppm methyl acetylene.

5. The method of claim 1 wherein said C₃₋ overhead stream directed to said inlet has a temperature ranging from about 160° to about 210° F.

6. The method of claim 5 wherein said hydrogenation reactor is operated at conditions comprising from about 10000 to about 18000 weight hourly space velocity and from about 250 to about 450 psig.

7. The method of claim 5 wherein said C₃₋ overhead stream directed to said inlet comprises from about 200 ppm to about 400 ppm CO, from about 0.1 ppm to about 10 ppm acetylene, from about 0 ppm to about 40 ppm propadiene, and from about 0 to about 40 ppm methyl acetylene.

8. The method of claim 1 wherein said C₃₋ overhead stream has a molar ratio of carbon monoxide/acetylene ranging from about 100 to about 20.

9. The method of claim 1 wherein said C₃₋ overhead stream has a molar ratio of carbon monoxide/acetylene ranging from about 80 to about 40.

10. The method of claim 1 wherein said fractionating takes place in a deetherizer fractionating tower which separates C₃ hydrocarbons from dimethyl ether and heavier boiling materials.

11. The method of claim 1 wherein said fractionating takes place in a depropanizer fractionating tower, which separates C₃ hydrocarbons and dimethyl ether from C₄ and heavier boiling materials.

12. The method of claim 1 wherein said fractionating takes place in a depropylenizer fractionating tower, which separates C₃⁼ and lighter boiling materials from propane and heavier boiling materials.

13. The method of claim 1 wherein at least about 95% of said acetylene is converted in said hydrogenation reactor.

14. The method of claim 1 wherein at least about 99% of said acetylene is converted in said hydrogenation reactor.

15. The method of claim 1 wherein said C₃₋ overhead stream directed to said inlet comprises acetylene, methyl acetylene and propadiene.

16. The method of claim 15 wherein at least about 95% of said acetylene, at least about 60% of said methyl acetylene and at least about 20% of said propadiene are converted in said hydrogenation reactor.

17. The method of claim 15 wherein at least about 99% of said acetylene, at least about 80% of said methyl acetylene and at least about 25% of said propadiene are converted in said hydrogenation reactor.

18. The method of claim 10 wherein an effluent from said hydrogenation reactor is directed to a demethanizer which removes hydrogen, carbon monoxide and methane from said effluent to provide a demethanizer product effluent.

19. The method of claim 11 wherein an effluent from said hydrogenation reactor is directed to a demethanizer which removes hydrogen, carbon monoxide and methane from said effluent to provide a demethanizer product effluent.

20. The method of claim 12 wherein an effluent from said hydrogenation reactor is directed to a demethanizer which removes hydrogen, carbon monoxide and methane from said effluent to provide a demethanizer product effluent.

21. The method of claim 18 wherein said demethanizer product effluent is directed to a C₂ splitter to provide an ethylene product stream comprising less than about 0.3 vppm acetylene.

22. The method of claim 18 wherein said demethanizer product effluent is directed to a C₃ splitter to provide a propylene product stream comprising less than about 2.0 vppm acetylene, less than about 3.0 vppm methyl acetylene and less than about 3.0 vppm propadiene.

23. The method of claim 19 wherein said demethanizer product effluent is directed to a C₂ splitter to provide an ethylene product stream comprising less than about 0.3 vppm acetylene.

24. The method of claim 19 wherein said demethanizer product effluent is directed to a C₃ splitter to provide a propylene product stream comprising less than about 2.0 vppm acetylene, less than about 3.0 vppm methyl acetylene and less than about 3.0 vppm propadiene.

25. The method of claim 20 wherein said demethanizer product effluent is directed to a C₂ splitter to provide an ethylene product stream comprising less than about 0.3 vppm acetylene.

26. The method of claim 20 wherein said demethanizer product effluent is directed to a C₃ splitter to provide a propylene product stream comprising less than about 2.0 vppm acetylene, less than about 3.0 vppm methyl acetylene and less than about 3.0 vppm propadiene.

27. The method of claim 1 wherein said olefinic stream contains an oxygenate impurity and is treated to at least partially remove said oxygenate impurity prior to said fractionating.

28. The method of claim 27 wherein said oxygenate impurity comprises dimethyl ether.

29. The method of claim 1 wherein said olefin stream from the hydrogenation reactor contains water and is directed to a molecular sieve dryer which provides a dried olefin stream from which water is at least partially removed.

30. The method of claim 1 wherein said olefin stream from the hydrogenation reactor contains water and methanol and is directed to a molecular sieve dryer which provides a dried olefin stream from which water and methanol are at least partially removed.

31. The method of claim 27 wherein said olefin stream from the hydrogenation reactor contains water and is directed to a molecular sieve dryer which provides a dried olefin stream from which water is at least partially removed.

32. The method of claim 27 wherein said olefin stream from the hydrogenation reactor contains water and methanol and is directed to a molecular sieve dryer which provides a dried olefin stream from which water and methanol are at least partially removed.

33. The method of claim 1 wherein said hydrogenation catalyst comprises a metal selected from the group consisting of Ni, Pd and Pt.

34. The method of claim 33 wherein said hydrogenation catalyst further comprises a metal selected from the group consisting of Cu, Ag and Au.

35. The method of claim 33 wherein said hydrogenation catalyst comprises an inorganic oxide support.

36. The method of claim 35 wherein said inorganic oxide support is alumina.

37. The method of claim 1 wherein said hydrogenation catalyst comprises palladium.

38. The method of claim 1 wherein said hydrogenation catalyst comprises palladium and silver, supported on calcium carbonate.

39. The method of claim 1 wherein said hydrogenation catalyst comprises palladium supported on alumina.

40. The method of claim 1 wherein said hydrogenation catalyst comprises from about 0.001 to about 2 wt % of said hydrogenation metal.

41. The method of claim 39 wherein said hydrogenation catalyst comprises from about 0.01 to about 1 wt % palladium.

43. The method of claim 1 wherein external hydrogen is added to said hydrogenation reactor.

44. The method of claim 1 wherein no external hydrogen is added to said hydrogenation reactor.

45. A method for converting oxygenates to olefins which comprises:
a) contacting an oxygenates feed in an oxygenates to olefins reactor with an oxygenates to olefins catalyst under conditions sufficient to provide an oxygenates to olefins product stream comprising ethylene, propylene, C₄ olefin, hydrogen, carbon monoxide, and acetylene;
b) fractionating said oxygenates to olefins product stream to provide a fractionated overhead stream comprising ethylene, propylene, hydrogen, from about 100 ppm to about 2000 ppm CO, from about 0.1 ppm to about 40 ppm acetylene, from about 0 ppm to about 40 ppm propadiene, and from about 0 to about 40 ppm methyl acetylene;
c) hydrogenating said fractionated overhead stream by contacting with a hydrogenation catalyst in a hydrogenation reactor under conditions sufficient to hydrogenate substantially all of said acetylene to olefin, without substantially hydrogenating said ethylene and said propylene; and
d) removing a purified olefin stream from the hydrogenation reactor.

45. The method of claim 44 wherein said fractionated overhead stream comprises from about 200 ppm to about 400 ppm CO, from about 0.1 ppm to about 10 ppm acetylene, from about 0 ppm to about 40 ppm propadiene, and from about 0 to about 40 ppm methyl acetylene.

46. The method of claim 44 wherein said fractionated overhead stream has a molar ratio of carbon monoxide/acetylene ranging from about 100 to about 20.

47. The method of claim 44 wherein said fractionated overhead stream has a molar ratio of carbon monoxide/acetylene ranging from about 80 to about 40.

48. The method of claim 44 wherein said fractionated overhead stream comprises propane.

49. The method of claim 44 wherein said fractionated overhead stream hydrogenated by said hydrogenation reactor has a temperature ranging from about 110° to about 250° F.

50. The method of claim 49 wherein said hydrogenation reactor is operated at conditions comprising from about 9000 to about 25000 weight hourly space velocity and from about 150 to about 500 psig.

51. The method of claim 44 wherein said fractionated overhead stream hydrogenated by said hydrogenation reactor has a temperature ranging from about 160° to about 210° F.

52. The method of claim 51 wherein said hydrogenation reactor is operated at conditions comprising from about 10000 to about 18000 weight hourly space velocity and from about 250 to about 450 psig.

53. The method of claim 44 wherein said fractionating takes place in a deetherizer fractionating tower which separates C₃ hydrocarbons from dimethyl ether and heavier boiling materials.

54. The method of claim 44 wherein said fractionating takes place in a depropanizer fractionating tower which separates C₃ hydrocarbons and dimethyl ether from C₄ and heavier boiling materials.

55. The method of claim 44 wherein said fractionating takes place in a depropylenizer fractionating tower which separates C₃⁼ from propane and heavier boiling materials.

56. The method of claim 44 wherein said fractionating takes place in a deetherizer, depropanizer, or depropylenizer.

57. The method of claim 56 wherein the purified olefin stream from said hydrogenation reactor contains water and is directed to a molecular sieve dryer which provides a dried olefin stream from which water is at least partially removed.

58. The method of claim 56 wherein the purified olefin stream from said hydrogenation reactor contains water and methanol and is directed to a molecular sieve dryer which provides a dried olefin stream from which water and methanol are at least partially removed.

59. The method of claim 57 wherein the dried olefin stream is cryogenically processed to provide a C₂ and C₃ fuel stream, a C₁ and hydrogen tail gas stream, an ethylene product stream and a propylene product stream.

60. The method of claim 59 wherein said ethylene product stream comprises less than about 0.3 vppm acetylene.

61. The method of claim 59 wherein said propylene product stream comprises less than about 2.0 vppm acetylene, less than about 3.0 vppm methyl acetylene and less than about 3.0 vppm propadiene.

62. The method of claim 45 wherein said hydrogenation catalyst comprises a metal selected from the group consisting of Ni, Pd and Pt.

63. The method of claim 62 wherein said hydrogenation catalyst further comprises a metal selected from the group consisting of Cu, Ag and Au.

64. The method of claim 62 wherein said hydrogenation catalyst comprises an inorganic oxide support.

65. The method of claim 64 wherein said inorganic oxide support is alumina.

66. The method of claim 45 wherein said hydrogenation catalyst comprises palladium.

67. The method of claim 45 wherein said hydrogenation catalyst comprises palladium and silver, supported on calcium carbonate.

68. The method of claim 45 wherein said hydrogenation catalyst comprises palladium supported on alumina.

69. The method of claim 45 wherein said hydrogenation catalyst comprises from about 0.001 to about 2 wt % of said hydrogenation metal.

70. The method of claim 45 wherein said hydrogenation catalyst comprises from about 0.01 to about 1 wt % palladium.

71. The method of claim 45 wherein external hydrogen is added to said hydrogenation reactor.

72. The method of claim 45 wherein no external hydrogen is added to said hydrogenation reactor.

73. The method of claim 45 wherein said oxygenates to olefins catalyst comprises a molecular sieve.

74. The method of claim 73 wherein said molecular sieve has a pore diameter of less than 5.0 Angstroms.

75. The method of claim 74 wherein said molecular sieve is selected from the group consisting of AEI, AFT, APC, ATN, ATT, ATV, AWW, BIK, CAS, CHA, CHI, DAC, DDR, EDI, ERI, GOO, KFI, LEV, LOV, LTA, MON, PAU, PHI, RHO, ROG, THO, ALPO-18, ALPO-34, SAPO-17, SAPO-18, SAPO-34, and substituted groups thereof.

76. The method of claim 75 wherein said molecular sieve is selected from the group consisting of ALPO-18, ALPO-34, SAPO-17, SAPO-18, and SAPO-34.

77. The method of claim 76 wherein said molecular sieve is SAPO-34.

78. The method of claim 73 wherein said molecular sieve has a pore diameter of 5-10 Angstroms.

79. The process of claim 78 wherein said molecular sieve is selected from the group consisting of MFI, MEL, MTW, EUO, MTT, HEU, FER, AFO, AEL, TON, and substituted groups thereof.

80. An apparatus for converting oxygenates to an olefins stream containing C₂ to C₄ olefins and acetylene as an impurity, and providing a purified ethylene and/or propylene stream proportionally reduced in said impurity content, said apparatus comprising:
i) an oxygenates to olefins reactor comprising a fluidized bed which comprises an oxygenates to olefins catalyst, said reactor further comprising an inlet for oxygenate feed and an outlet for said olefins stream;
ii) a fractionator for separating from said olefins stream a bottoms stream containing unreacted oxygenate, C₄₊ hydrocarbons and waste water, and an overheads stream comprising ethylene, propylene, hydrogen, acetylene and CO;
iii) a hydrogenation reactor for hydrogenating said overheads stream by contacting with a hydrogenation catalyst under conditions sufficient to hydrogenate substantially all of said acetylene to olefin, without substantially hydrogenating said ethylene and said propylene, to provide a purified stream of reduced acetylene content; and
iv) a means for cryogenically fractionating said purified stream to provide a purified ethylene product and a purified propylene product.

81. The apparatus of claim 80 wherein said fractionator is a fractionating tower, a deetherizer which separates C₃ hydrocarbons from dimethyl ether and heavier boiling materials.

82. The apparatus of claim 80 wherein said fractionator is selected from the group consisting of deetherizer, depropanizer, and depropylenizer.

83. The apparatus of claim 80 wherein said fractionator is a deetherizer fractionating tower which separates C₃ hydrocarbons from dimethyl ether and heavier boiling materials.

84. The apparatus of claim 80 wherein said fractionator is a depropanizer fractionating tower which separates C₃ hydrocarbons and dimethyl ether from propane and heavier boiling materials.

85. The apparatus of claim 80 wherein said fractionating takes place in a depropylenizer fractionating tower which separates C₃⁼ from propane and heavier boiling materials.

86. The apparatus of claim 80 which further comprises a means for quenching said olefins stream to provide a quenched olefins stream.

87. The apparatus of claim 86 which further comprises a means for compressing said quenched olefins stream to provide a compressed, quenched olefins stream.

88. The apparatus of claim 80 which further comprises a caustic treater for treating said overheads stream to remove carbon dioxide from said overheads stream to provide a caustic-treated stream.

89. The apparatus of claim 88 which further comprises a molecular sieve dryer upstream from said hydrogenation reactor, to remove water from said caustic-treated stream.

90. The apparatus of claim 88 which further comprises a molecular sieve dryer downstream from said hydrogenation reactor, to remove water from said purified stream of reduced acetylene content.

91. The apparatus of claim 88 which further comprises a molecular sieve dryer downstream from said hydrogenation reactor, to remove water and methanol from said purified stream of reduced acetylene content.
